(19) United States
(12) Patent Application Publication
(10) Pub. No.: US 2018/0371551 A1
(43) Pub. Date: Dec. 27, 2018

(54) MAT2A INHIBITORS FOR TREATING MTAP NULL CANCER

(71) Applicant: Agios Pharmaceuticals, Inc., Cambridge, MA (US)

(72) Inventors: Katya Marjon, Sharon, MA (US); Sung Eun Choe, Lexington, MA (US)

(21) Appl. No.: 15/780,494

(22) PCT Filed: Dec. 2, 2016
(86) PCT No.: PCT/US2016/064619

§ 371 Date: May 31, 2018

Related U.S. Application Data
(60) Provisional application No. 62/297,572, filed on Feb. 19, 2016.
(60) Provisional application No. 62/262,750, filed on Dec. 3, 2015.

(51) Int. Cl. C12Q 1/6886; A61P 35/00; A61K 31/505; A61K 31/44; A61K 31/136

(52) U.S. Cl. CPC C12Q 1/6886; A61P 35/00; C12Q 2600/156; A61K 31/44; A61K 31/136; A61K 31/505

(57) ABSTRACT

The present invention provides diagnostic and prognostic methods for predicting the effectiveness of treatment of a cancer patient with a MAT2A inhibitor. Methods are provided for predicting the sensitivity of tumor cell growth to inhibition by a MAT2A inhibitor, comprising assessing whether the tumor cell is absent an MTAP gene whereby cells that are MTAP null are sensitive to inhibition by MAT2A inhibitors.

### FIELD OF THE INVENTION

The present invention is directed to methods for treating and diagnosing cancer patients. In particular, the present invention is directed to methods for determining which patients will benefit from treatment with inhibitor of methionine adenosyltransferase (MAT2A).

### BACKGROUND OF THE INVENTION

The identification and characterization of oncogenic gain-of-function mutations and their corresponding molecular pathways has spurred the development of a number of targeted therapies that provide substantial benefit to cancer patients with the corresponding mutation. This includes drugs selective for cancers driven by gain-of-function point mutations (such as erlotinib and gefitinib in mutant EGFR non-small cell lung cancer (Lynch & Haber, NEJM 2004 and Pao & Varmus PNAS 2004)), genomic amplifications (such as trastuzumab in HER2-amplified breast cancer (Slamon and Norton NEJM 2001)), or oncogenic gene fusions (such as imatinib in BCR-ABL-positive chronic myelogenous leukemia (Druker & Sawyers NEJM 2001)). In each case, the therapy directly inhibits the oncogenic mutant protein, abrogating its function. Loss-of function mutations in tumor suppressor genes are highly prevalent, and equally important in the molecular pathogenesis of cancer, yet there are very few examples of therapies that selectively target cancers on the basis of loss-of-function mutations in tumor suppressors (Morris & Chan Cancer 2015). This discord can be explained by the simple observation that the mutant protein cannot be directly inhibited for therapeutic benefit. Tumor suppressors that are inactivated by homozygous deletion are most problematic for targeted therapy, since the lack of residual protein obviates therapeutic strategies that would directly activate, stabilize, or repair the defective tumor suppressor.

Methionine adenosyltransferase (MAT) also known as S-adenosylmethionine synthetase is a cellular enzyme that catalyzes the synthesis of S-adenosyl methionine (SAM or AdoMet) from methionine and ATP and is considered the rate-limiting step of the methionine cycle. SAM is the propylamino donor in polyamine biosynthesis and the principal methyl donor for DNA methylation and is involved in gene transcription and cellular proliferation as well as the production of secondary metabolites.

Two genes, MAT1A and MAT2A, encode two distinct catalytic MAT isoforms. A third gene, MAT2B, encodes a MAT2A regulatory subunit. MAT1A is specifically expressed in the adult liver, whereas MAT2A is widely distributed. Because MAT isoforms differ in catalytic kinetics and regulatory properties, MAT1A-expressing cells have considerably higher SAM levels than do MAT2A-expressing cells. It has been found that hypomethylation of the MAT2A promoter and histone acetylation causes upregulation of MAT2A expression.

In hepatocellular carcinoma (HCC), the downregulation of MAT and the up-regulation of MAT2A occur, which is known as the MAT1A:MAT2A switch. The switch accompanied with up-regulation of MAT2B results in lower SAM contents, which provide a growth advantage to hepatoma cells. Because MAT2A plays crucial role in facilitating the growth of hepatoma cells, it is a target for antineoplastic therapy. Recent studies have shown that silencing by using small interfering RNA substantially suppress growth and induce apoptosis in hepatoma cells.

Methylthioadenosine phosphorylase (MTAP) is an enzyme found in all normal tissues that catalyzes the conversion of methylthioadenosine (MTA) into adenine and 5-methylthioribose-1-phosphate. The adenine is salvaged to generate adenosine monophosphate, and the 5-methylthioribose-1-phosphate is converted to methionine and formate. Because of this salvage pathway, MTA can serve as an alternative purine source when de novo purine synthesis is blocked, e.g., with antimetabolites, such as L-alanosine.

Many human and murine malignant cells lack MTAP activity. MTAP deficiency is not only found in tissue culture cells but the deficiency is also present in primary leukemias, gliomas, melanomas, pancreatic cancers, non-small cell lung cancers (NSLC), bladder cancers, astrocytomas, osteosarcomas, head and neck cancers, myxoid chondrosarcomas, ovarian cancers, endometrial cancers, breast cancers, soft tissue sarcomas, non-Hodgkin lymphomas, and mesothelionmas. The gene encoding for human MTAP maps to region 9p21 on human chromosome 9p. This region also contains the tumor suppressor genes p16^{INK4A} (also known as CDKN2A), and p15^{INK4B}. These genes code for p16 and p15, which are inhibitors of the cyclin D-dependent kinases cdk4 and cdk6, respectively.

The p16^{INK4A} transcript can alternatively be ARF spliced into a transcript encoding p14^{ARF}. p14^{ARF} binds to MDM2 and prevents degradation of p53 (Pomerantz et al. (1998) Cell 92:713-723). The 9p21 chromosomal region is of interest because it is frequently homozygously deleted in a variety of cancers, including leukemias, NSLC, pancreatic cancers, gliomas, melanomas, and mesothelioma. The deletions often inactivate more than one gene. For example, Cairns et al. ((1995) Nat. Gen. 11:210-212) reported that after studying more than 500 primary tumors, almost all the deletions identified in such tumors involved a 170 kb region containing MTAP, p14ARF and P16INK4A Carson et al (WO 99/67634) reported that a correlation exists between the stage of tumor development and loss of homozygosity of the gene encoding MTAP and the gene encoding p16. For example, deletion of the MTAP gene, but not p16^{INK4A} was reported to be indicative of a cancer at an early stage of development, whereas deletion of the genes encoding for p16 and MTAP was reported to be indicative of a cancer at a more advanced stage of tumor development. Garcia-Castellano et al reported that in some osteosarcoma patients, the MTAP gene was present at diagnosis but was deleted at a later time point (Garcia-Castellano et al., supra).

### SUMMARY OF THE INVENTION

The present invention provides a method for treating a cancer in a subject wherein said cancer is characterized by reduction or absence MTAP expression or absence of the MTAP gene or reduced function of MTAP protein said method comprising administering to the subject a therapeutically effective amount of a MAT2A inhibitor.

The present invention provides a method for determining whether survival or proliferation of a tumor cell can be inhibited by contacting said tumor cell with a MAT2A inhibitor, said method comprising determining the status of MTAP in said tumor cell, wherein the reduction or absence MTAP expression or absence of the MTAP gene or reduced level or function of MTAP protein indicates survival or proliferation of said tumor cell can be inhibited by a MAT2A inhibitor.

In another aspect, the present invention provides a method for characterizing a tumor cell comprising measuring in said tumor cell the level of MTAP gene expression, the presence or absence of an MTAP gene or the level of MTAP protein present, wherein the reduction or absence MTAP expression or absence of the MTAP gene or reduced level or function of MTAP protein relative to a reference cell indicates that survival or proliferation of said tumor cell can be inhibited by a MAT2A inhibitor.

In another aspect, the present invention provides a method of determining the responsiveness of a tumor to MAT2A inhibition comprising determining in a sample of said tumor a reduced expression level of an MTAP gene, the absence of an MTAP gene or reduction of the level or function of MTAP protein, wherein a reduced expression level of an MTAP gene, the absence of an MTAP gene or reduction of the level or function of MTAP protein indicates said tumor is responsive to a MAT2A inhibitor.

In another aspect, the present invention provides a kit comprising a reagent for measuring in a tumor sample the expression level of an MTAP gene, the absence of an MTAP gene or reduction of the level or function of MTAP protein, said kit further comprising instructions for administering a therapeutically effective amount of a MAT2A inhibitor.

### BRIEF DESCRIPTION OF THE FIGURES

FIGS. 1A-F. Functional Genomics Screening Identifies Genes that are Synthetic Lethal with MTAP loss. Schematic depicting chromosome 9 and 9p21.3 region containing MTAP gene in close proximity to CDKN2A genomic region encompassing p16 INK4A/p14/ARF genes. (B) Schematic depicting shRNA depletion screen in colon carcinoma HCT116 MTAP wt and MTAP^{-/-} isogenic cell line pair. (C) Immunoblot analysis demonstrating a lack of MTAP protein expression in HCT116 MTAP^{-/-} cells. (D) Gene scores in HCT116 MTAP^{-/-} vs. MTAP wt cells. The gene score was calculated as SUM log 2 fold change in the abundance of each of the 8 shRNAs targeting that gene in HCT116 MTAP^{-/-} cells vs. HCT116 MTAP wt cells at the end of cell culture period vs. prior to introduction to cells. (E) Top 10 genes that scored as differentially depleted in the MTAP-deficient HCT116 cells. Genes pursued in subsequent studies are highlighted in green (MAT2A), red (PRMT5), and magenta (RIOK1). (F) Changes in the abundance of the individual MAT2A, PRMT5, and RIOK1 shRNAs in HCT116 MTAP^{-/-} vs. HCT116 wt cells in the screen. Individual shRNAs are highlighted in green (MAT2A), red (PRMT5), or magenta (RIOK1). The rest of the shRNAs in the library are shown as grey diamonds.

FIGS. 2A-F. PRMT5 is selectively essential in MTAP-null cells upon genetic ablation but not pharmacologic targeting. Immunoblot analysis of the indicated proteins in HCT116 MTAP^{-/-} and HCT116 MTAP wt cells stably expressing PRMT5 shRNA and p-LVX empty vector control (EV). (B) PRMT5 is selectively essential in MTAP-null cells in vitro. Percent growth of HCT116 wt and HCT116 MTAP^{-/-} cells upon PRMT5 knockdown (+dox), with or without PRMT5 wt or R368A mutant rescue, versus no knockdown (no dox) control in a 10-day soft agar colony growth assay. Colonies were stained with crystal violet and then quantified using Li-Cor (mean±SD, n=3). (C) Immunoblot analysis of the indicated proteins in HCT116 MTAP^{-/-} and HCT116 MTAP wt cells stably expressing PRMT5 shRNA and shRNA-resistant PRMT5 wt cDNA or PRMT5 R368A catalytically-dead mutant cDNA. (D) Immunoblot analysis of symmetric di-methylarginine marks in HCT116 MTAP^{-/-} and HCT116 MTAP wt cells stably expressing PRMT5 shRNA and p-LVX empty vector control (EV), or PRMT5 shRNA and shRNA-resistant PRMT5 wt cDNA or PRMT5 R368A catalytically-dead mutant cDNA. (E) Dose response analysis with EPZ015666 titrated from 20 μM top dose in HCT116 MTAP wt vs. HCT116 MTAP^{-/-} cells. Cells were treated with EPZ015666 for 5 days and their response to the compound is measured as fold growth of treated cells vs. untreated control (mean±SD, n=3). (F) Immunoblot analysis of PRMT5-dependent di-methylarginine marks in HCT116 isogenic pair treated with indicated doses of EPZ015666 for 5 days. HCT116 wt and HCT116 MTAP^{-/-} cells expressing PRMT5 shRNA were used as a control and PRMT5 knockdown was induced with doxycycline for 6 days. Dox indicates where doxycycline (200 ng/ml) was added for 6 days to induce PRMT5 shRNA expression prior to cell collection and immunoblot analysis.

FIGS. 3A-D. MTA Accumulates in MTAP-deficient cancers. Schematic of methionine recycling and salvage pathways. MTAP is the enzyme in methionine salvage pathway that converts methylthioadenosine (MTA), a byproduct of polyamines biosynthesis, from decarboxylated S-adenosylmethionine (dcSAM) and Putrescine, back to methionine and adenine. MTAP deletion results in accumulation of its substrate MTA that is inhibitory to the activity of methyltransferases, enzymes mediating one-carbon methyl group (CH3) transfer from SAM. SAM is generated by MAT2A in cells. S-adenosylhomocysteine (SAH) is generated as a byproduct of methyl transfer reactions and it is recycled back to methionine via re-methylation of homocysteine. Alternatively, homocysteine is converted to cysteine and is directed into transsulfuration pathway generating glutathione. (B) Intracellular metabolite levels analysis using un-targeted LC-MS in HCT116 isogenic pair. Waterfall plot demonstrates the log 2 of mean fold change (FC) in HCT116 MTAP^{-/-} cells compared to HCT116 wt control vs. metabolite ID. Volcano plot of the log 2 of mean fold change (FC) in HCT116 MTAP^{-/-} cells compared to HCT116 wt control vs. log₁₀ p value for each metabolite is also shown. MTA and dcSAM are highlighted in red. (C) Quantitative measurement of intracellular MTA levels in HCT116 isogenic cell lines (mean±SD, n=3). (D) Media MTA levels in a panel of 249 cancer cell lines of various tumor origin.

FIGS. 4A-E. MTA inhibits PRMT5 activity in vitro and in vivo. (A) MTA sensitivity of a panel of N-methyltransferases. A panel of small molecule, DNA, as well as lysine and arginine N-methyltransferases was tested using an in vitro assay in presence of 10 and 100 μM concentrations of MTA. (B) Dose response curve for MTA inhibition of PRMT5 complex activity in an in vitro assay. (C) PRMT5 is the most sensitive to inhibition by MTA among all methyltransferases tested. Waterfall plot of the MTA Ki values is shown and PRMT5 data point is highlighted in red. (D) MTAP deletion reduces basal activity of PRMT5 in cells. Immunoblot analysis of the indicated proteins in a panel of MTAP wt and MTAP-deleted cancer cell lines of various tumor origin. HCT116 wt and HCT116 MTAP^{-/-} cell lines were included as a reference. Levels of H4R3me2s marks were quantified using Li-Cor software, normalized to the total levels of histone H4, and average value±SEM was reported on the bar graph. p value was calculated using 2-tailed unpaired t-test. (E) MTAP pharmacologic inhibition with 5-methylthioadenosine transition state analogue inhibitor (MTAPi) leads to the reduction in symmetric di-methylarginine marks in HCT116 wt cells. Immunoblot analysis of the indicated proteins in HCT116 MTAP^{-/-} cells and HCT116 MTAP wt cells treated with MTAP inhibitor at 250 or 500 nM for 3 days,

FIGS. 5A-J. MAT2A is selectively essential in MTAP-null HCT116 cells. Immunoblot analysis of the indicated proteins in HCT116 MTAP^{-/-} and HCT116 MTAP wt cells stably expressing non-targeting shRNA (shNT), MAT2A shRNA, MAT2A shRNA and shRNA-resistant MAT2A wt cDNA (+Resc), or MAT2A shRNA and MTAP cDNA (+MTAP). Dox indicates where doxycycline (200 ng/ml) was added for 7 days to induce MAT2A shRNA expression prior to cell collection and analysis. (B) MAT2A knockdown in vitro results in equal SAM depletion in HCT116 wt and HCT116 MTAP^{-/-} cells. SAM levels were measured using targeted LC-MS analysis in the HCT116 isogenic pair expressing inducible shMAT2A with (+dox) and without (−dox) MAT2A knockdown. (C) MAT2A is selectively essential in MTAP-deficient HCT116 cells in vitro. Percent growth of HCT116 wt and HCT116 MTAP^{-/-} cells upon MAT2A knockdown (+dox), with or without MAT2A wt (+Resc) or MTAP (+MTAP) rescue, versus no knockdown (−dox) control measured in a 4- and 6-day in vitro growth assay (mean±SD, n=5). Cells were pre-treated with 200 ng/ml dox for 4 days prior to plating for a growth assay. (D) Immunoblot analysis of the indicated proteins in HCT116 MTAP wt and HCT116 MTAP^{-/-} xenografts stably expressing MAT2A shRNA. Dox indicates where doxycycline (2,000 mg/kg) was added to the mouse chow to induce MAT2A shRNA expression. (E) MAT2A knockdown in vivo results in equal SAM depletion in HCT116 wt and HCT116 MTAP^{-/-} xenografts. SAM levels were measured using targeted LC-MS analysis in xenografts formed from the HCT116 isogenic pair expressing inducible shMAT2A with (dox) or without (no dox) MAT2A knockdown. (F) MAT2A is selectively essential in MTAP-deficient HCT116 cells in vivo. Kinetics of tumor growth upon in vivo ablation of MAT2A in subcutaneous xenografts of shMAT2A HCT116 isogenic pair cell lines. Doxycycline treatment was initiated once tumors reached 200-300 mm³ in diameter (mean±SEM, n=5-6). (G) Growth of MTAP-deficient HCT116 cells in vivo upon MAT2A knockdown is rescued by MAT2A wt cDNA. Kinetics of tumor growth upon in vivo ablation of MAT2A in subcutaneous xenografts of HCT116 MTAP^{-/-} cell lines stably expressing shNT, shMAT2A, or shMAT2A and hairpin-resistant MAT2A cDNA. Doxycycline treatment was initiated once tumors reached 200-300 mm³ in diameter (mean±SEM, n=5-6). (H) Immunoblot analysis of the indicated proteins in HCT116 MTAP^{-/-} xenografts stably expressing shNT, shMAT2A, or shMAT2A and hairpin-resistant MAT2A cDNA. Dox indicates where doxycycline (2,000 mg/kg) was added to the mouse chow to induce MAT2A shRNA expression. (I) MAT2A is essential in MTAP-deleted MCF7 cells in vitro. Percent growth of MCF7 cells upon MAT2A knockdown (+dox), with or without MAT2A wt (+Resc) rescue, versus no knockdown (−dox) control measured in a 7-day in vitro growth assay (mean±SD, n=5). (J) Immunoblot analysis of the indicated proteins in MCF7 cells stably expressing non-targeting shRNA (shNT), MAT2A shRNA, MAT2A shRNA and shRNA-resistant MAT2A wt cDNA (+Resc). Dox indicates where doxycycline (200 ng/ml) was added for 7 days to induce MAT2A shRNA expression prior to cell collection and analysis.

FIGS. 6A-C. MAT2A ablation selectively inhibits PRMT5 activity in MTAP-null cells. PRMT activity is reduced upon genetic ablation of MAT2A. Immunoblot analysis of the indicated proteins was performed in the HCT116 isogenic cell lines stably expressing non-targeting shRNA (shNT), MAT2A shRNA, MAT2A shRNA and shRNA-resistant MAT2A wt cDNA (+Resc), or MAT2A shRNA and MTAP cDNA (+MTAP). Dox indicates where doxycycline (200 ng/ml) was added for 7 days to induce MAT2A shRNA expression prior to cell collection and analysis. (B) PRMT5 exhibits the lowest affinity for SAM. SAM Km values (□M) were plotted for all methyltransferases analyzed for their sensitivity to inhibition by MTA. (C) Schematic depicting convergence of MTAP deficiency-induced metabolic vulnerability due to MTA accumulation and reduced levels of SAM upon MAT2A ablation upon PRMT5, resulting in reduced PRMT5 function in MTAP-deleted, SAM-deprived environment.

FIGS. 7A-D. Multiple PRMT5 co-complexes are vulnerable in MTAP-null cells. Immunoblot analysis of the indicated proteins in HCT116 MTAP^{-/-} and HCT116 MTAP wt cells stably expressing RIOK1 shRNA, RIOK1 shRNA and empty vector control (EV), RIOK1 shRNA and shRNA-resistant RIOK1 wt cDNA (wt RIOK1) or RIOK1 K208R/D324N catalytically-dead mutant cDNA. Dox indicates where doxycycline (200 ng/ml) was added for 6 days to induce PRMT5 shRNA expression prior to cell collection and analysis. (B) RIOK1 is selectively essential in MTAP-null cells in vitro. Percent growth of HCT116 wt and HCT116 MTAP^{-/-} cells upon RIOK1 knockdown (dox), with or without RIOK1 wt or RIOK1 K208R/D324N mutant (RIOK1mut) rescue, versus no knockdown (no dox) control in a 10-day soft agar colony growth assay. Colonies were stained with crystal violet and then quantified using Li-Cor (mean±SD, n=3). (C) Additional PRMT5-binding partners are selectively essential in MTAP-null cells. Percent growth of HCT116 wt and HCT116 MTAP^{-/-} cells upon transfection with non-targeting siRNA (NT), or siRNA targeting PRMT5, RIOK1, pICln, MEP50, COPR5, or SMRACA4 normalized to NT control as measured in a 4-day growth assay following two rounds of transfection with siRNA pools (mean±SD, n=5). (D) qPCR confirmation of PRMT5 and PRMT5 binding partners knockdown using siRNA pools. Knockdown efficiencies were calculated relative to the levels of mRNA detected in non-targeting (NT) siRNA pool-transfected cells.

FIGS. 8A-B. (A) Percent growth inhibition of MTAP null and MTAP wild type HCT116 cells treated with MAT2A inhibitor AGI-512. (B) Percent growth inhibition of MTAP null an dMTAP wildtype HCT116 cells treated with MAT2A inhibitor AGI-673.

FIG. 9. Immunoblot analysis of PRMT5, MTAP and beta-actin proteins and SDMA marks in HCT116 MTAP−/− and MTAPwt cells.

FIG. 10. Effect of Mat2a knockdown in in vivo orthotopic MCF7 model.

FIGS. 11A-D. PRMT5 is a selective vulnerability in MTAP-null cancers

FIG. 12. MAT2A depletion reduces PRMT5 methyl marks in MTAP null cells.

### DETAILED DESCRIPTION

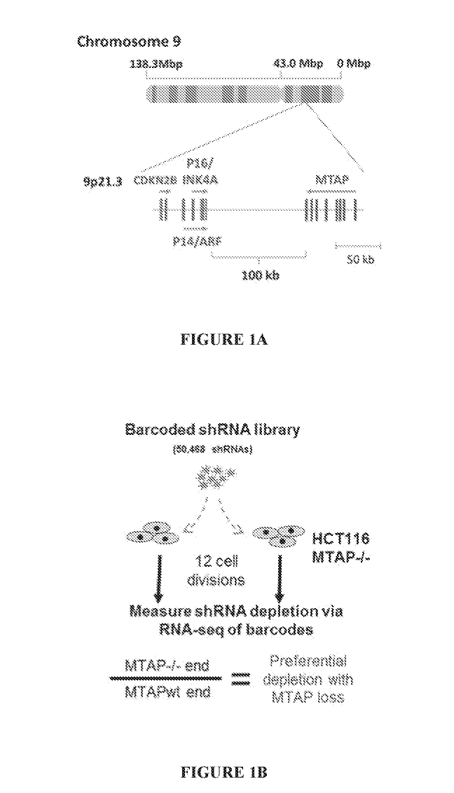

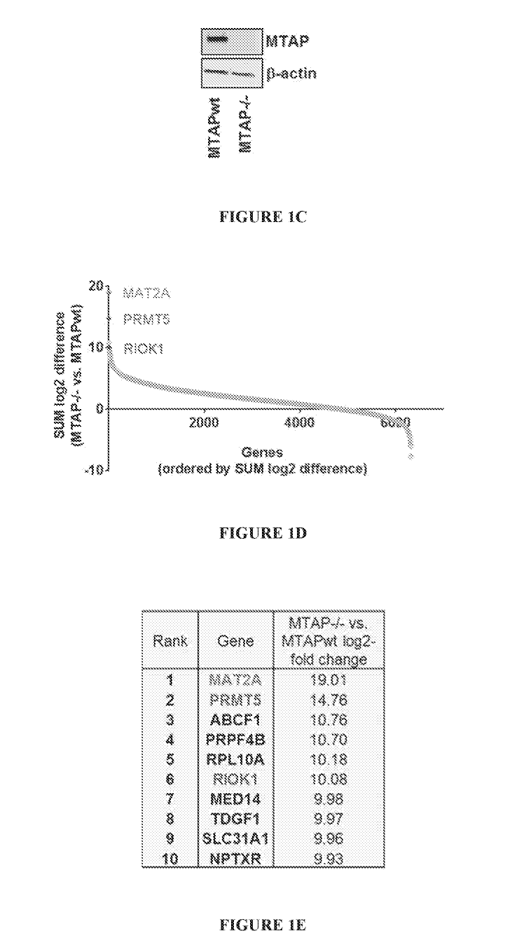

Chromosome 9p21 (Chr9p21) is homozygous deleted in approximately 15% of all human cancer (Berhoukim Meyerson nature 2010), including a number of different tumor types and ranging in frequency up to the >50% deletion frequency observed in Glioblastoma Multiforme (Parsons and Kinsler, Science 2008). The 9p21 locus includes the CDKN2a gene, which encodes both p14-ARF and p16-INK4a (FIG. 1A). Both proteins have tumor suppressive roles, with p14-ARF known to stabilize p53 (Kamijo & Sherr Cell 1997 and Zhang & Yarbough Cell 1998) and p16-INK4a demonstrated to be a critical cell cycle regulator and potent tumor suppressor via negative regulation of the CDK4/6 cell cycle kinases (Serrano & Beach Nature 1993). Although Chr9p21 deletion was first discovered over 30 years ago (Chilcote NEJM 1985), molecularly targeted therapies for CDKN2A loss have proven elusive, and it may be necessary to identify alternative approaches to target tumors with deletion of Chr9p21.

Notably, Chr9p2l deletions frequently involve co-deletion of genes proximal to CDKN2A (FIG. 1A). Foremost among these co-deleted genes is MTAP, which resides on Chr9p21 adjacent to CDKN2a (FIG. 1A). The MTAP gene is within 100 kb of CDKN2A, and homozygous deletion of MTAP is found in 80-90% of tumors with CDKN2A deletion (Illie & Ladanyi Clin Canc Res 1993 and Zhang & Savarese Canc Genet Cytogenet 1996). MTAP encodes Methylthioadenosine Phosphorylase, a critical enzyme in the methionine salvage pathway. MTAP metabolizes the byproduct of polyamine synthesis, methylthioadenosine, leading to the eventual regeneration of methionine and adenine from MTA (Zappia & Cartena-Farrina Adv Exp Med Biol 1988). Thus MTAP resides at the intersection of methionine metabolism, polyamine biosynthesis, and nucleotide metabolism—metabolic pathway s that are each important in the proliferative metabolism of cancer cells. In fact, MTAP deletion has been reported to create sensitivity to inhibitors of purine biosynthesis (Li and Bertino Oncol Res 2004), although this metabolic vulnerability is lost in vivo, as tumors uptake circulating adenine and escape the purine biosynthesis sensitivity (Rueffli-Brasse and Wickramasinghe JCI 2011). We sought to ask whether MTAP deletion creates other targetable collateral vulnerabilities in cancers with Chr9p21 deletion.

To screen for vulnerabilities that arise upon MTAP loss in cancer, shRNA depletion screening was used in an isogenic cancer cell line pair that vary only in MTAP status. Although MTAP encodes a metabolic enzyme, we hypothesized that MTAP loss may create collateral vulnerabilities in biologic pathways that extend beyond metabolism. Precedent for such cross-talk between metabolic and non-metabolic pathways includes the observation that the metabolite 2-hydroxyglutarate, produced by gain-of-function mutant IDH1/2 proteins, can inhibit members of the alpha-ketoglutarate dependent dioxygenase enzyme family (Xu & Xiong Cancer Cell 2011, Rohle & Mellinghoff Science 2013). A similar mechanism has also been implicated in tumors with mutations in Succinate Dehydrogenase (SDH) or Fumarate Hydratase (FH), where the substrates of those enzymes accumulate to high levels (Selak and Gottlieb, Cancer Cell 2005 and Issacs and Neckers, Cancer Cell 2005). Thus, aberrations in the cancer metabolome can impinge on non-metabolic pathways. To test the hypothesis that MTAP deletion would create collateral vulnerabilities in metabolic and non-metabolic pathways, an shRNA library was used consisting of shRNA hairpins targeting the 3000+ genes of the metabolome as well as an additional 3000+ additional non-metabolic genes.

Through this screen and subsequent investigation, a signaling axis was identified that becomes vulnerable upon MTAP loss in cancer. Central in this signaling axis is the arginine methyltransferase, PRMT5. Using metabolomic and biochemical approaches, it was discovered that MTA, the substrate of the MTAP enzyme reaction, accumulates in MTAP-null cancers. MTA inhibits PRMT5 enzyme activity and leads to reduced basal PRMT5 methylation in MTAP-null cancers. This vulnerability extends both upstream and downstream of PRMT5. We show that the metabolic enzyme Methionine-adenosyltransferase-2A (MAT2A), which produces PRMT5 substrate S-adenosyl methionine (SAM), is also selectively essential in MTAP-null cancers, as are multiple different PRMT5 binding partners, including the kinase RIOK1.

shRNA Depletion Screen in HCT116 MTAP Wt/MTAP^{-/-} Isogenic Pair.

In order to identify genes whose loss would lead to selective killing of MTAP-deficient cells, an shRNA-based depletion screen were performed in HCT116 colon carcinoma cell line and an isogenic clone of HCT116 cells that had been genetically modified to delete exon 6 of the MTAP gene (FIG. 1B). This deletion led to complete loss of MTAP protein expression (FIG. 1C). To provide broad coverage for potential synthetic lethal interactions, we constructed a library that encompassed the complete metabolome (3,067 genes), the mitochondrial proteome (Pagliarini and Mootha Cell 2008), the epigenome (Arrowsmith and Shapira Nature Reviews Drug Discovery 2012), the kinome (http://www.uniprot.orgi), and >1500 additional genes representing diverse biologic pathways. HCT116 MTAP^{-/-} and HCT116 wt cells were transduced with the shRNA library containing 8 shRNAs per gene, and the pool of knockdown cells was passaged for 12 cell divisions. At the end of the culture we measured the relative abundance of each shRNA barcode via deep sequencing, and calculated the fold depletion of each shRNA compared to the untransduced library DNA. We then calculated an MTAP selectivity score for each gene based on the difference in the log 2 fold change in the abundance of each of the 8 shRNAs targeting the gene in HCT116 MTAP^{-/-} vs. HCT116 wt cells (FIG. 1D).

This analysis demonstrated that while the majority of the genes, as well as shRNA controls, had a similar score in both HCT116 MTAP^{-/-} and HCT116 MTAP wt cells (FIG. 1D), a subset of genes was selectively depleted in MTAP-deficient cells (FIG. 1D-E). The top hit in the screen was MAT2A, which encodes the metabolic enzyme Methionine Adenosyltransferase II, alpha (FIG. 1D-F). MAT2A catalyzes the synthesis of the universal biological donor of methyl groups, S-adenosylmethionine (SAM) via adenosylation of methionine. The second best scoring gene in the screen was Protein Arginine Methyltransferase 5 (PRMT5) (FIG. 1D-F), which is the catalytic subunit of a multiprotein methyltransferase complex that includes PRMT5 in complex with obligate binding partner WD45/MEP50 (WD repeat domain 45/methylosome protein 50), and other scaffolding proteins (Meister et al., 2001; Pesiridis et al., 2009). PRMT5 belongs to the type II PRMT subfamily of arginine methyl transferases and catalyzes the formation of symmetric di-methylarginines in target proteins. Interestingly, the sixth highest scoring gene, RIOK1, encodes a Rio domain containing protein, which is a binding partner of PRMT5 that directs PRMT5 towards selective methylation of a subset of PRMT5 substrates (Guderian et al 2011). These data suggest that MAT2A and PRMT5-catalyzed reactions are critical for maintaining viability of MTAP-deficient cells. Although all three highlighted hits represent therapeutically and biologically interesting targets, we initially focused our attention on PRMT5 as there are currently ongoing efforts targeting this enzyme for the treatment of human cancer (Chan Penebre Nature Chem Bio 2015).

### PRMT5 is Selectively Essential in MTAP-Null Cells Upon Genetic Ablation but not Pharmacologic Targeting.

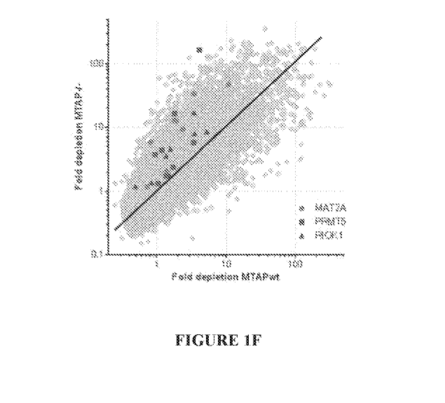

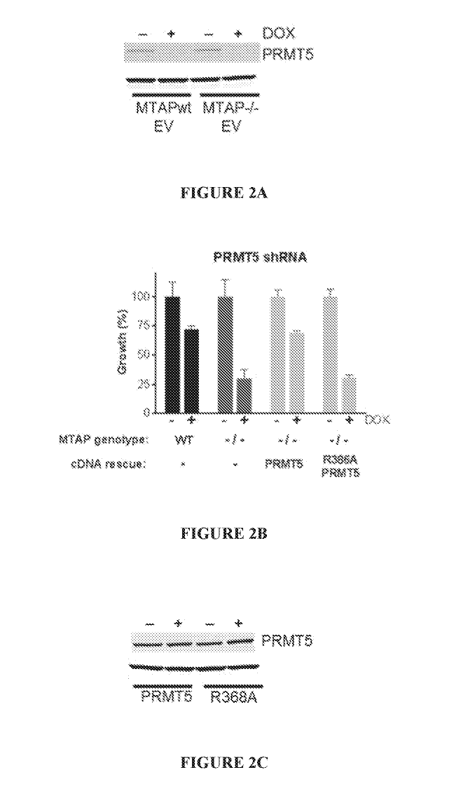

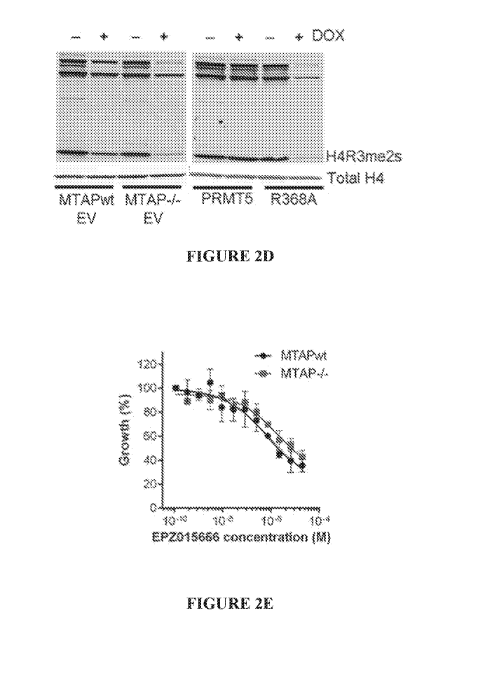

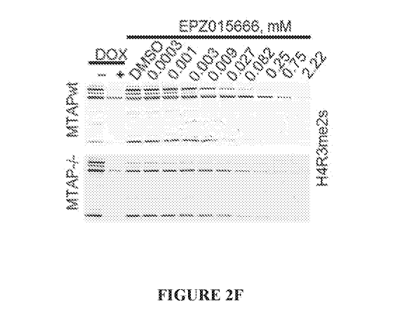

To further investigate the connection between MTAP deficiency and PRMT5 function in cells, we generated HCT116 MTAP^{-/-} and HCT116 wt cell lines stably expressing inducible shRNAs targeting PRMT5. We confirmed that PRMT5 was efficiently knocked down by measuring levels of PRMT5 protein. Consistent with our gnomic screening results, PRMT5 knockdown with doxycycline-inducible shRNA led to more complete growth reduction in cells with MTAP deletion than in MTAP WT cells (FIG. 2B). Expression of an shPRMT5-resistant PRMT5 cDNA in the MTAP-null cells rescued growth inhibition upon endogenous PRMT5 knockdown, while expression of catalytically-dead R368A PRMT5 mutant (Pollack et al., 1999) cDNA did not (FIG. 2B-C). Thus, the anti-proliferative effects of our shRNA are due to PRMT5 depletion and not due to off-target shRNA effects. Lack of rescue by R386A-mutant PRMT5 indicates that PRMT5 enzyme activity is essential in MTAP−/− cells. Interestingly, equivalent reduction in PRMT5 protein level in MTAP−/− and WT cells resulted in greater reduction in the levels of symmetric di-methylarginine marks in the MTAP−/− cell line and was rescued by PRMT5 but not R368A-mutant cDNA (FIG. 2D). These findings provide validation of our screening results and further suggest that PRMT5 catalytic function is critical for maintaining growth of MTAP-deficient cells.

Next we wanted to interrogate PRMT5 function in MTAP-deficient cells using a pharmacologic tool. A potent and selective inhibitor of PRMT5, EPZ015666, was recently developed (Chan-Penebre et al., 2015). We utilized the EPZ015666 compound and performed dose response analysis in the HCT116 isogenic pair (FIG. 2E). However, unlike genetic targeting of PRMT5, growth inhibition upon pharmacologic targeting of PRMT5 was not selective for the MTAP-deficient genetic background (FIG. 2E). This finding was unexpected considering that the catalytically-dead mutant of PRMT5 did not rescue the growth phenotype in HCT116 MTAP^{-/-} cells, suggesting it is the loss of catalytic function of PRMT5 that was necessary to selectively inhibit the growth of these cells (FIG. 2A). Interestingly, unlike with genetic ablation of PRMT5 function, equal degree of PRMT5 activity inhibition was achieved in both MTAP^{-/-} and wt HCT116 cells with EPZ015666, as evidenced by reduced levels of PRMT5-dependent di-methylarginine marks in total cell lysates (FIG. 2F). This surprising discrepancy between impact of genetic and pharmacologic PRMT5 ablation on the growth of MTAP-deficient cells led us to further interrogate the basic biology and metabolism behind PRMT5 and MTAP.

### MTAP Deficiency Creates an Altered Metabolic State.

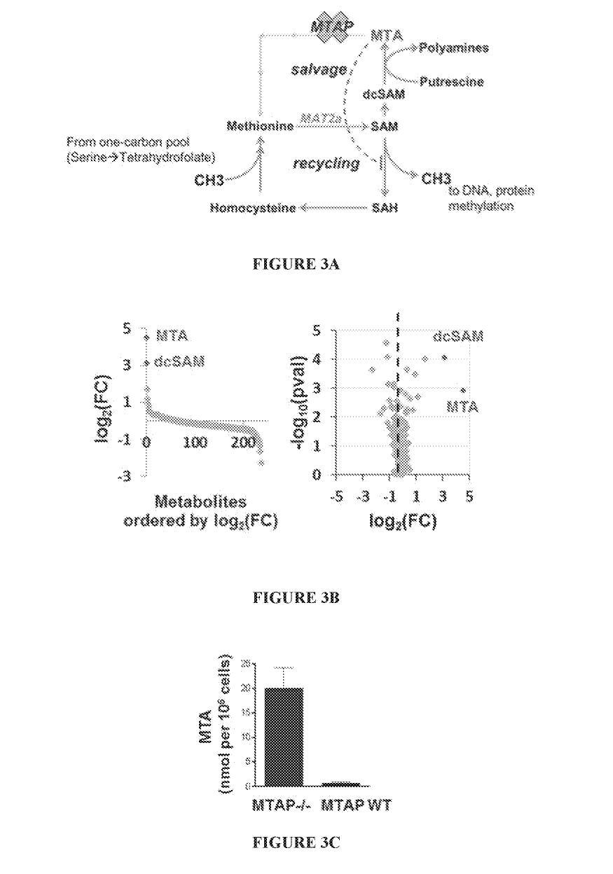

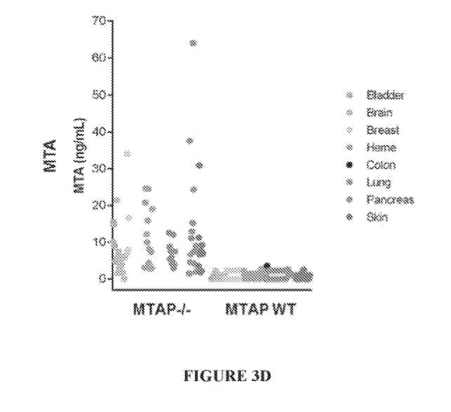

In order to explain the difference in the impact of genetic vs. pharmacologic targeting of PRMT5 on growth of HCT116 isogenic pair, we wanted to further build our mechanistic understanding of MTAP and PRMT5 synthetic lethality, MTAP is an enzyme in the methionine salvage pathway that converts a byproduct of polyamine biosynthesis, methylthioadenosine (MTA), back to methionine and adenine (FIG. 3A). Since MTAP is the only enzyme in mammalian cells known to catalyze the degradation of MTA, we hypothesized that MTAP deficiency would result in accumulation of MTA. We first tested this hypothesis in the context of a broader, untargeted LC-MS based metabolomics assessment of intracellular metabolite levels in the HCT116 MTAP isogenic pair (FIG. 3B). This analysis revealed that, among 237 annotated metabolites that were detected, MTA displayed the largest increase in HCT116 MTAP^{-/-} cells compared to HCT116 wt control. Interestingly, decarboxylated S-adenosylmethionine (dcSAM), the metabolite upstream from MTA in the polyamine biosynthetic pathway, displayed the second-largest increase. The enrichment of these two metabolites in HCT116 MTAP^{-/-} cells was highly statistically-significant (FIG. 3B). Elevation of MTA was further confirmed using quantitative measurement of MTA levels in HCT116 isogenic pair (FIG. 3C). Furthermore, a screen of a large cancer cell line panel comprising 249 cell lines of different tumor origin demonstrated very consistent accumulation of MTA in the media of cells with endogenous MTAP deletion (FIG. 3D).

### MTA Inhibits PRMT5 Activity In Vitro and In Vivo.

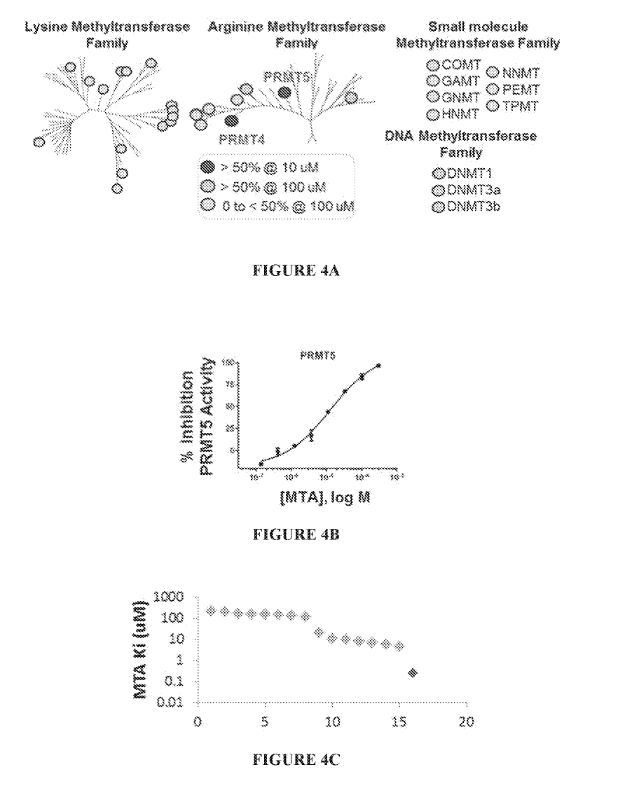

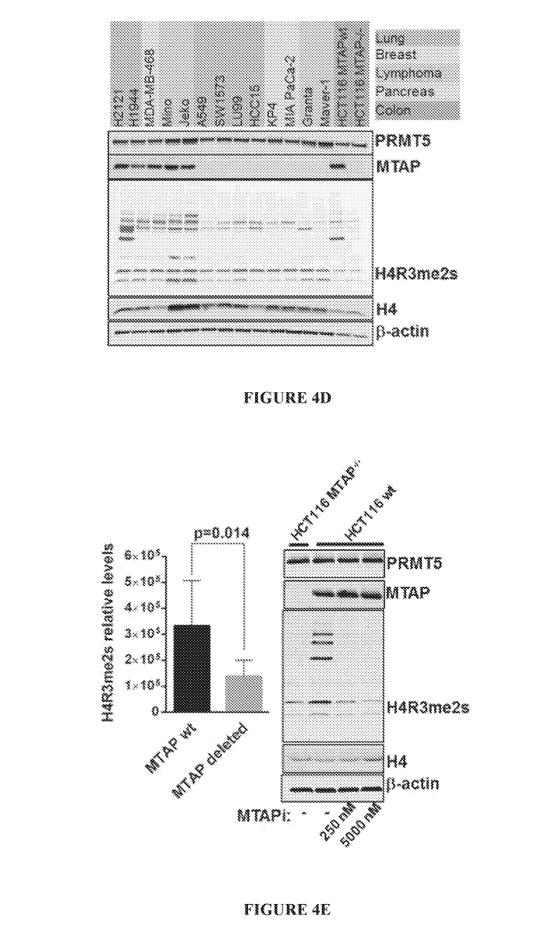

MTA has been reported to inhibit activity of protein methyltransferases (Enouf et al., 1979). To test this notion directly, we performed an in vitro biochemical screen assessing enzyme activity of 33 different N-methyltransferases following treatment with 10 μM and 100 μM MTA (FIG. 4A). Inhibition by MTA was only observed in a small subset of the panel, and strongest inhibition was observed for PRMT5 and PRMT4, members of the arginine methyltransferase family (FIG. 4A). Further, PRMT5 demonstrated potent sensitivity to MTA in subsequent experiments testing a wide range of MTA concentrations (FIG. 4B). Next, we analyzed the MTA Ki for PRMT5, PRMT4, and a diverse subset of methyltransferases (FIG. 4C).

Strikingly, the MTA Ki for PRMT5 (0.46 μM) was >20-fold lower than that for any other methyltransferase, indicating that PRMT5 is far more sensitive to inhibition by MTA than any other methyltransferase tested. This biochemical observation is consistent with our shRNA screening data demonstrating that PRMT5 was the strongest hit among all the methyltransferases that were represented in the library and were selectively depleted in HCT116 MTAP^{-/-} cells (FIG. 1D).

We next addressed the impact of MTA accumulation on PRMT5 activity in cells. According to our LC-MS analysis of intracellular MTA levels in MTAP-deficient cells (˜100 μM) and PRMT5 IC₅₀ for MTA measured in our biochemical assay (3 μM), we hypothesized that MTA accumulation in MTAP-deficient cells would be sufficient to result in inhibition of PRMT5 activity. During our analysis of PRMT5-dependent methyl marks in total cell lysates of the HCT116 isogenic pair, we noted that HCT116 MTAP^{-/-} cells appeared to have lower basal levels of methylation (FIG. 2D). To further substantiate this finding, we performed western blot analysis of PRMT5-dependent methyl marks in total cell lysates of a subset of MTAP wt and MTAP-deleted cell lines (FIG. 4D). We observed that MTAP-deleted cell lines consistently demonstrated lower levels of symmetric di-methylarginine marks (FIG. 4D). Finally, we took advantage of the availability of a potent, cell permeable transition state analogue inhibitor of MTAP (Basu et al., 2011; Longshaw et al., 2010). We treated HCT16 wt cells with the MTAP inhibitor for three days and measured the impact of pharmacologic inhibition of MTAP on the levels of di-methylarginine marks (FIG. 4E). Treatment with MTAP inhibitor at the dose sufficient to increase MTA levels to those observed in HCT116 MTAP^{-/-} cells (Figure S4) resulted in reduction in the levels of di-methylarginine methyl marks similarly to what is observed upon genetic ablation of MTAP (FIG. 4E). These data strongly indicate that PRMT5 activity is impaired by MTA in MTAP-null cells, resulting in reduced methylation of its protein substrates and creating a vulnerability to additional reduction of PRMT5 activity by shRNA. Furthermore, our finding that MTA inhibits of PRMT5 provides an explanation for the lack of MTAP-selective growth inhibition with the PRMT5 inhibitor EPZ015666. This inhibitor binds selectively to the SAM-PRMT5 complex (Chan-Penebre et al., 2015) via a cation-pi molecular interaction that is not possible with the MTA-PRMT5 complex. Since MTA prevents binding of SAM to PRMT5, and EPZ015666 only interacts with SAM-bound PRMT5, MTA binding is mutually exclusive with EPZ015666 binding. Two inhibitors of a single enzyme can only be synergistic if they bind to separate binding sites and their interaction with target is not mutually exclusive (Breitinger).

### MAT2A is Selectively Essential in MTAP-Deficient Cells.

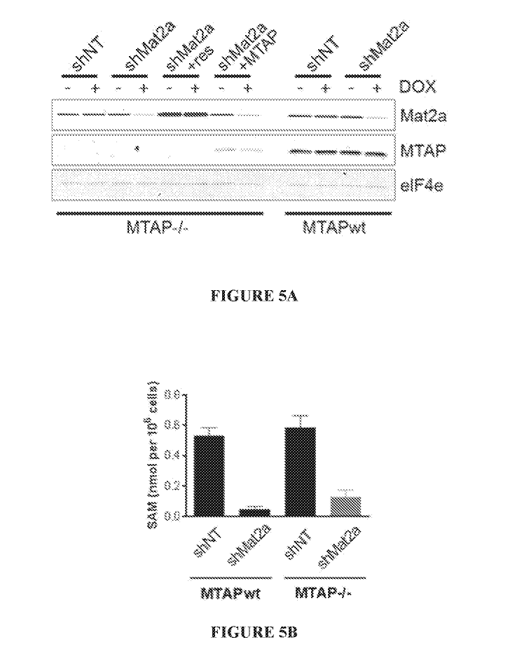

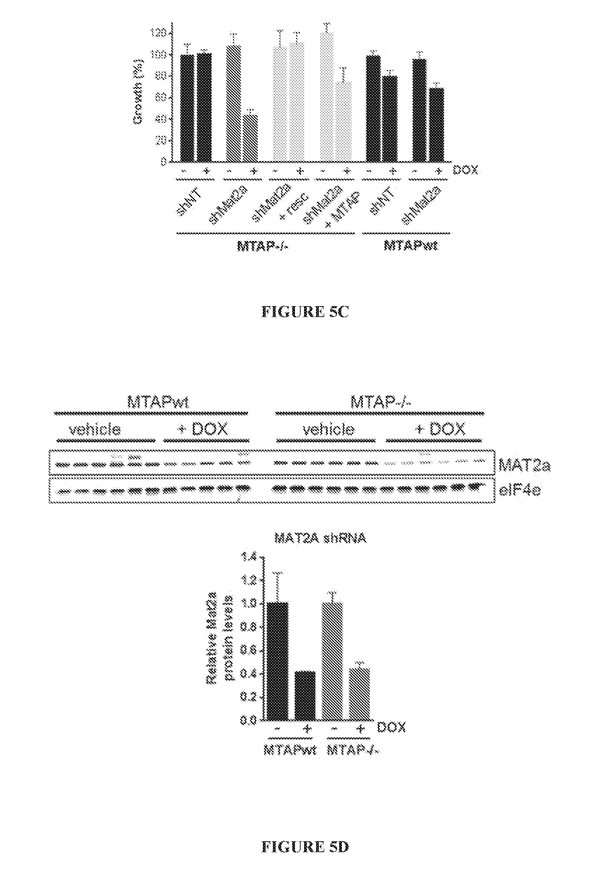

We next wanted to test whether MAT2A, the top hit in our shRNA screen, also represents a bona fide synthetic lethal target in MTAP-deficient cells. We thus utilized the HCT116 isogenic pair and created cell lines stably expressing non-targeting shRNA, MAT2A-targeting shRNA, as well as cell lines that were additionally reconstituted with shRNA-resistant MAT2A cDNA, or that expressed MTAP cDNA. We confirmed efficient MAT2A knockdown, and MAT2A and MTAP re-expression in HCT116 cells by western blot (FIG. 5A). We also confirmed that MAT2A knockdown resulted in reduced cellular levels of SAM in both HCT116 genotypes using LC-MS analysis (FIG. 5B). We further confirmed that MTAP re-expression depleted high MTA levels present in the media of HCT116 MTAP^{-/-} cells. We then tested the impact of MAT2A knockdown in HCT116 wt vs. HCT116 MTAP^{-/-} cells in a 4- and 6-day in vitro growth assay (FIG. 5C). The results were in agreement with our genomic screen. MAT2A knockdown selectively attenuated growth of HCT116 MTAP^{-/-}, but not HCT116 wt cells (FIG. 5C). Importantly, this growth defect was rescued by introduction of shRNA-resistant MAT2A cDNA construct, indicating on-target effect of the shRNA, and was also partially rescued by MTAP re-expression (FIG. 5C).

To investigate the in vitro to in vivo translation of our findings, we conducted xenograft efficacy studies with HCT116 isogenic cell lines expressing inducible MAT2A shRNA. In these studies, tumors were allowed to form prior to treatment of animals with doxycycline, to assess the role of MAT2A in proliferation of established tumors. Efficiency of MAT2A knockdown in vivo was confirmed by western blot (FIG. 5D). We further confirmed that MAT2A genetic ablation in vivo resulted in a similar reduction in SAM levels in HCT116 xenografts of both genotypes (FIG. 5E). In accordance with our findings in vitro, MTAP-selective growth inhibition was observed in vivo upon MAT2A depletion by shRNA (FIG. 5F). To demonstrate that this selective growth inhibition in vivo was an on-target effect, we performed expanded in vivo study with a wild type MAT2A rescue arm of shMAT2A (FIGS. 5G and 5H). This experiment confirmed the efficacy observed in our first in vivo study (FIGS. 5G and 5H) and, as with the in vitro studies, growth inhibition was rescued in the xenograft expressing a MAT2A cDNA that was resistant to the MAT2A shRNA (FIGS. 5G and 5H).

Finally, we wanted to confirm our findings in a model that possesses endogenous deletion in MTAP locus. Thus, we generated breast carcinoma MCF7 cell lines stably expressing non-targeting shRNA, MAT2A-targeting shRNA, as well as cell lines that were additionally reconstituted with shRNA-resistant MAT2A cDNA. We demonstrated efficiency of MAT2A knockdown and re-expression by western blot (FIG. 5J). Consistent with observations made in the HCT116 model system, MAT2A knockdown attenuated growth of MTAP-deleted MCF7 cells in a 7-day growth assays (FIG. 5I), while MAT2A cDNA reconstitution resulted in complete rescue of the growth phenotype. Thus, MAT2A demonstrates consistent synthetic lethality with MTAP deficiency in our models.

### MAT2A Loss Selectively Inhibits PRMT5 Activity in MTAP Null Cells.

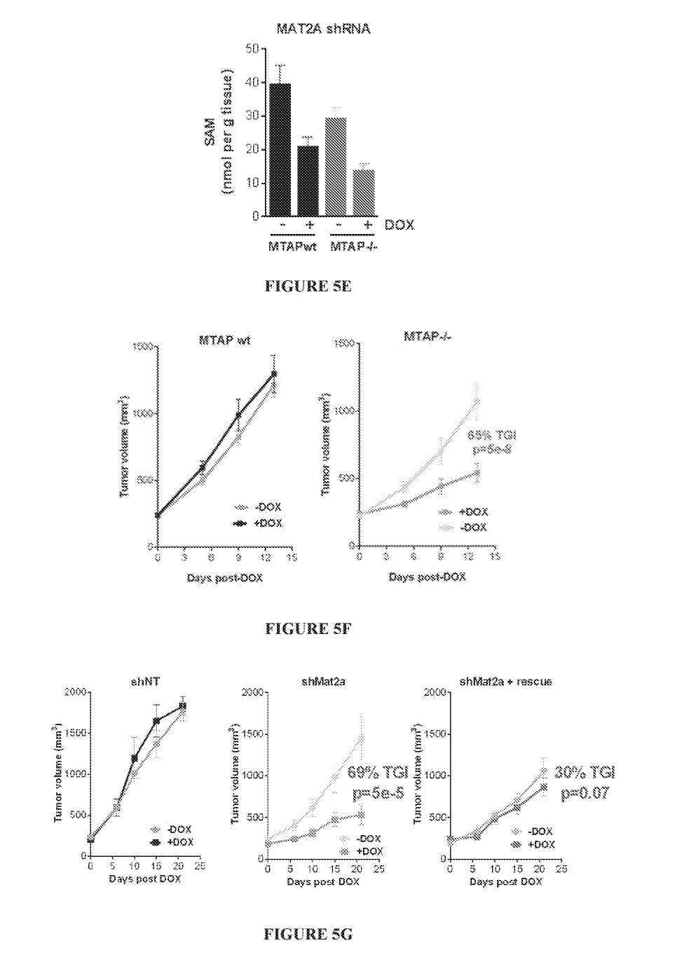

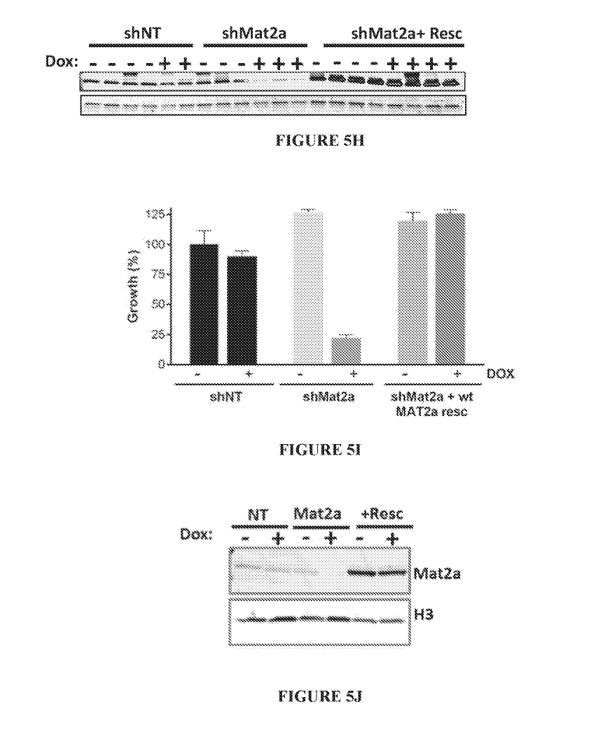

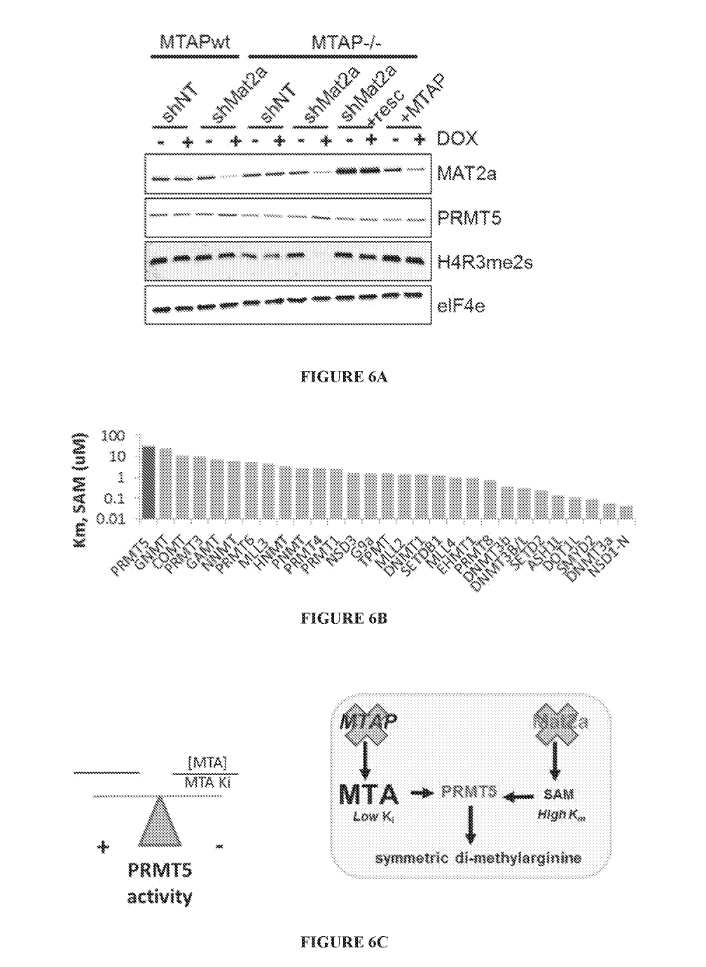

Having confirmed that both PRMT5 and MAT2A are true synthetic lethal partners of MTAP, we wanted to assess whether there is a mechanistic link between these two top hits in our screen. Indeed, MAT2A generates SAM, which is necessary for the activity of all cellular methyltransferases, and reduction in SAM levels upon MAT2A genetic ablation would be expected to broadly impact their function, including that of PRMT5. Thus, we measured levels of PRMT5-dependent symmetric di-methylarginine marks on histone H4 in our MAT2A shRNA HCT116 isogenic pair, as well as in MAT2A reconstituted and MTAP re-expression cell lines upon knockdown of MAT2A (FIG. 6A). Interestingly, we observed that despite equivalent degree of reduction in SAM levels in HCT116 cells of both genotype (FIG. 5B), H4R3me2s marks were selectively reduced in MTAP-deficient cells, but not in MTAP wt cells, and were rescued in presence of MAT2A and MTAP cDNA (FIG. 6A). In combination with our observation regarding the strong inhibitory impact of MTA on PRMT5 activity, these data suggest that PRMT5 function in the MTAP-null background is highly dependent on adequate availability of SAM. PRMT5 was reported in the literature to exhibit low affinity for SAM (Antonysamy et al., 2012; Sun et al., 2011), We thus compared SAM Km values for the N-methyltransferases from our in vitro biochemical panel analysis and observed that indeed PRMT5 exhibited the lowest affinity for SAM (FIG. 6B). This finding may explain PRMT5 dependence on proper MAT2A function, especially in the metabolically-altered, high-MTA environment of MTAP-deficient cells (FIG. 6C). Thus, metabolic vulnerability due to MTAP deficiency extends upstream of PRMT5 creating dependence on the availability of PRMT5 substrate SAM and therefore the activity of SAM-producing enzyme MAT2A.

### Multiple PRMT5 Co-Complexes are Vulnerable in MTAP-Null Cells.

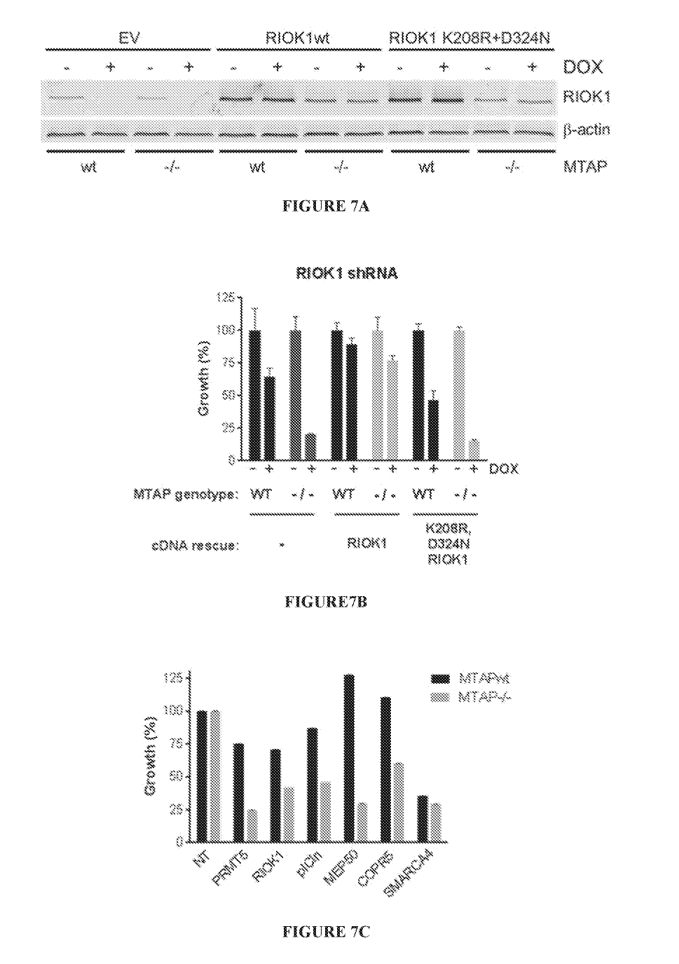

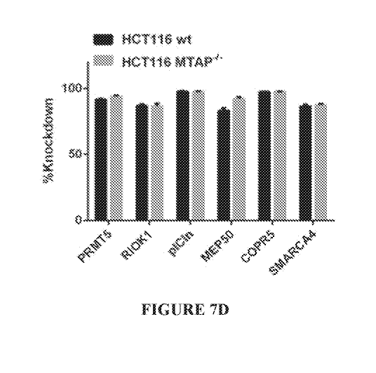

The Rio domain containing protein RIOK1 was another strong hit in our shRNA depletion screening campaign. Since it is a PRMT5 binding partner, we sought to confirm the synthetic lethal phenotype upon genetic ablation of RIOK1 in the HCT116 MTAP isogenic cells. Similar to the characterization that was performed for PRMT5 and MAT2A, inducible RIOK1 sh-RNA cell lines, as well as RIOK1 wt rescue and RIOK1 active site (D324N) and ATP-binding domain (K208R) catalytically inactive mutant (Angermayr et al., 2002; Widmann et al., 2012) cell lines were created. RIOK1 knockdown and re-expression efficiencies were evaluated by western blot (FIG. 7A). Confirming our finding in the genomic screening, RIOK1 knockdown resulted in a selective inhibition of growth of HCT116 MTAP^{-/-} cells with minimal impact on growth of HCT116 wt cells (FIG. 7B). The growth phenotype was rescued by the expression of shRNA-resistant wt RIOK1 and not catalytically inactive K208R, D324N mutant RIOK1 (FIG. 7B). These data suggest that the metabolic vulnerability created via accumulation of MTA in MTAP-deficient background further extends downstream of PRMT5 via impact on PRMT5 binding partner RIOK1.

PRMT5 participates in several multimeric protein co-complexes, including obligatory binding partner WD45/MEP50 (Wilczek et al., 2011), the mutually exclusive partners pICln and RIOK1 (Guderian et al., 2011), the nuclear regulator of specificity COPR5 (cooperator of PRMT5)(Lacroix et al., 2008), and others. Neither MEP50, nor pICln or other binding partners of PRMT5 were represented in our shRNA library. Thus, to evaluate the possibility that the vulnerability of MTAP-deficient cells further extends to PRMT5 co-complexes beyond the RIOK1 co-complex, we performed siRNA pool-mediated knockdown of multiple PRMT5 co-complex members, including PRMT5 itself, RIOK1, MEP50, pICln, and COPR5 in the HCT116 isogenic pair (FIGS. 7C and 7D). We observed the selective inhibition of the growth of MTAP-deficient cells upon knockdown of each member of the PRMT5 co-complex (FIG. 7C). Importantly, knockdown of a separate PRMT5-binding protein, the ATP-dependent helicase Brg1 encoded by the SMARCA4 gene, (Pal et al., 2004) inhibited growth of HCT116 cells regardless of their MTAP status (FIG. 7C). These data suggest that vulnerability of MTAP-deficient cells downstream from PRMT5 is not restricted to RIOK1 co-complex but is rather broad impacting several co-complexes involving PRMT5 as a binding partner. MTA accumulation in MTAP-null cells reduces PRMT5 activity and creates a collateral vulnerability to targeting of PRMT5. This vulnerability extends to the metabolic, epigenetic, and signaling pathway members that reside upstream, and downstream, of PRMT5.

The mammalian metabolome is characterized by a high degree of flexibility and redundancy (Thielle & Pallson Nat Biotech 2013 and Folger and Shlomi Molec Sys Bio 2011.). MTA is thus unusual in that it is consumed by a solitary, non-redundant enzyme, MTAP. We observed that upon MTAP deletion, MTA accumulates to an intracellular concentration of approximately 100 uM, and cells begin to excrete excess MTA. This accumulation of MTA led to an unexpected collateral vulnerability in the arginine methyltransferase PRMT5. While the shRNA library contained 39 methyltransferases, PRMT5 was unique in its high degree of MTAP-selectivity. Biochemical profiling of methyltransferases revealed a molecular basis for this phenomenon. Amongst the 32 methyltransferases that we tested in vitro, PRMT5 was the enzyme most sensitive to inhibition by MTA. In vitro inhibition of PRMT5 by MTA occurs at the concentrations very similar to those observed in MTAP-null cells, suggesting that this is a biologically-relevant phenomenon. Consistent with this, we observed substantially reduced basal levels of PRMT5 methyl marks in cells with MTAP deletion.

Reduced basal PRMT5 activity creates a vulnerability to further ablation of PRMT5 by shRNA. Interestingly, treatment with PRMT5 inhibitor EPZ-015666 did not lead to selective growth inhibition in MTAP-null cells. EPZ-015666 has a very distinctive mode of inhibition of PRMT5. This inhibitor is SAM-uncompetitive and forms key binding interactions with enzyme-bound SAM via an unusual cation-pi interaction with the partial positively charged methyl group on SAM (Chan-penebre Nat Chem Bio 2015). MTA is unable to form this synergistic binding interaction with EPZ-015666 (CITE Chan-penebre). Thus this existing PRMT5 inhibitor does not display preferential activity in MT AP-null cancers. Exploiting the PRMT5 vulnerability in MTAP-null cancers may require the development of MTA-selective PRMT5 inhibitors that bind to the MTA-bound form of PRMT5 and trap the enzyme in that state. MTA-selective inhibitors might afford a greater therapeutic window than non-selective inhibitors, as MTAP expression in normal tissues should provide a protective effect by maintaining low MTA levels. Mouse genetics studies have revealed that PRMT5 has important roles in normal physiology; PRMT5 knockout leads to embryonic lethality (Tee 2010), and substantial toxicities arise upon tissue specific PRMT5 knockout in the CNS (Bezzi 2013) skeletal muscle (Zhang 2015) and hematopoietic lineages (Liu 2015). These toxicities may become dose-limiting in the clinical setting, narrowing the therapeutic potential of agents that target PRMT5 in a non-selective manner.

Cellular methyltransferase activity is subject to regulatory control by small molecule metabolites. It has previously been established that methyltransferases are regulated by the relative balance of substrate SAM and product SAH (Vance Cui Biochim Biophys Acta 1997). The SAM/SAH ratio is used to calculate cellular ‘methylation potential’ as a measure of cellular poise to conduct methyltransferase reactions (Williams & Schalinske J Nutrition 2006). Our observation that PRMT5 can be inhibited by MTA implicates PRMT5 as the exemplar member of a biochemically-distinct family of methyltransferases that can be regulated by SAM/MTA ratio. This novel regulatory mode is revealed very clearly in MTAP-null cancer cells, where MTA levels accumulate dramatically. There exists only limited information regarding MTA levels across normal tissues (Stevens & Oefner, J chromatography 2010), and wider MTA screening might reveal other settings in which MTA accumulation leads to inhibition of PRMT5. We note also that PRMT5 has a fairly weak binding affinity for SAM. This is unusual amongst the methyltransferase family, as most mammalian methyltransferases have SAM Km values 10- to 100-fold below the physiologic concentration of SAM (Richon & Copeland Chem Biol Drug Design 2011). This biochemical finding implies that PRMT5 is poised as a SAM-sensitive methyltransferase, and this sensitivity is exemplified by the reduction in PRMT5 methyl marks that is observed upon MAT2A depletion in MTAP-null cells.

PRMT5 regulates a number of proliferative and biosynthetic processes, such as histone methylation that controls expression of cell cycle genes (Chung & Sif JBC 2013), methylation of growth factor signaling components like EGFR and Raf (Hsu & Hung Nat Cell Bio 2011, Andreu-Perez & Recio, Sci Signaling 2011), and methylation of key protein components required for maturation of ribosome and spliceosome complexes (Ren & Xu, JBC 2010, and Friesen & Dreyfuss Mol Cell Bio 2001). Thus PRMT5 activity leads to coordinated upregulation of a range of pro-proliferative and biosynthetic pathways. The vulnerability of PRMT5 in MTAP-deficient cancers extends both upstream of PRMT5S (to MAT2A) and downstream of PRMT5 (to RIOK1 and other PRMT5 cocomplex members). Collectively, these proteins comprise a metabolic-epigenetic-signaling axis which senses and transmits information about nutrient availability (MAT2A substrate Methionine) to the multiple biosynthetic pathways that reside downstream of PRMT5. This axis presents intriguing opportunities for targeted therapy of MTAP-deficient cancers. In addition to the potential to devise MTA-selective PRMT5 inhibitors, our work demonstrates that therapeutic targeting of MAT2A, RIOK1, or other PRMT5 co-complex members, could selectively impact MTAP-null cancers while sparing MTAP-expressing normal tissues. Thus this vulnerable axis includes a number of proteins that merit further consideration as therapeutic targets to address the ˜15% of human cancers with deletion of the MTAP/p16/CDKN2A locus.

Cell Line Screen with AGI-512 and AGI-673

AG-512 and AG-673 are small molecule inhibitors of MAT2A enzymatic activity demonstrating an IC₅₀ of 83 nM and 143 nM respectively in a biochemical assay and inhibited the production of SAM in cells with IC₅₀s of 80 and 490 nM respectively. These compounds were screened for growth inhibition against several cancer cell lines having varied tissue origin for which MTAP status (null or wild type) was determined. The results are presented in table 1.

TABLE 1

| | MTAP | | AGI-673 | AGI-512 |
|---|---|---|---|---|
| CELL LINE | STATUS | TISSUE | IC50 (μM) | IC50 (μM) |
| LN-18 | null | brain | 0.397 | 0.485 |
| HMCB | WT | skin | 0.465 | 0.473 |
| K-562 | null | heme | 0.596 | 0.901 |
| MDA-MB-231 | null | breast | 0.560 | 0.364 |
| SW 1088 | null | brain | 0.639 | 0.508 |
| GB-1 | null | brain | 1.302 | 3.844 |
| NCI-H1437 | null | lung | 2.020 | 0.695 |
| A172 | null | brain | 2.174 | 0.283 |
| A549 | null | lung | 3.052 | 3.647 |
| HCC70 | WT | breast | 3.156 | 2.162 |
| U-87 MG | null | brain | 4.001 | 2.115 |
| Jurkat | null | heme | 3.667 | 1.168 |
| MDA-MB-468 | WT | breast | 3.981 | 3.034 |
| NCI-H661 | WT | lung | 5.137 | 6.249 |
| HCT 116 | WT | colon | 10.165 | >20 |
| Hs 695T | WT | skin | 10.866 | >20 |
| NCI-H460 | WT | lung | 13.307 | >20 |
| COLO 741 | null | skin | 12.652 | 7.643 |
| CCF-STTG1 | WT | brain | >20 | >20 |
| NMC-G1 | WT | brain | 8.504 | >20 |
| LN-229 | WT | brain | 0.159 | 0.111 |
| RT-112 | null | bladder | 0.496 | 0.326 |
| H4 | null | brain | 0.774 | 0.886 |
| SW 780 | null | bladder | 0.375 | 0.628 |
| Hs 294T | WT | skin | 0.329 | 0.529 |
| RT4 | null | bladder | 6.373 | 0.566 |
| DLD-1 | WT | colon | 13.996 | 0.941 |
| U-251 MG | WT | brain | 2.822 | 13.412 |
| HT-1197 | WT | bladder | 0.889 | 0.761 |
| KNS-42 | WT | brain | >20 | >20 |
| YH-13 | null | brain | 0.823 | 0.485 |
| Daoy | null | brain | 1.030 | 0.852 |
| M059K | WT | brain | 0.401 | 1.116 |
| U-118 MG | null | brain | 0.759 | >20 |
| SNU-1105 | null | brain | 0.267 | 0.103 |
| SW 1783 | WT | brain | 3.605 | >20 |
| U251 | WT | brain | 1.681 | 0.511 |
| MV-4-11 | WT | heme | 2.307 | 5.741 |
| NCI-H1568 | WT | lung | 1.384 | 1.780 |
| MeWo | WT | skin | 1.101 | 1.281 |
| Hs 839.T | WT | skin | 0.269 | 4.068 |
| IGR-1 | null | skin | 0.044 | 0.023 |
| KS-1 | null | brain | 0.487 | 0.505 |
| COLO 829 | WT | skin | 1.762 | 2.229 |
| ONS-76 | null | brain | 2.794 | 0.794 |
| HS 683 | null | brain | 1.804 | 1.118 |
| DBTRG-05MG | WT | brain | 4.237 | 0.323 |
| SF126 | WT | brain | 6.357 | 8.273 |
| HT-144 | WT | skin | 7.693 | 2.255 |
| YKG-1 | WT | brain | 10.256 | 10.093 |
| Becker | WT | brain | 9.652 | >20 |
| U-138 MG | null | brain | 12.292 | >20 |
| GI-1 | WT | brain | 0.958 | 0.967 |
| KNS-60 | WT | brain | 15.640 | >20 |
| KNS-81 | WT | brain | 11.775 | 11.279 |
| J82 | WT | bladder | 1.634 | 1.900 |
| SCaBER | WT | bladder | 1.350 | 1.142 |
| T98G | WT | brain | 2.101 | 3.788 |
| KALS-1 | WT | brain | 1.589 | 1.822 |
| Hs 940.T | WT | skin | 0.737 | 0.514 |
| Hs 688(A).T | WT | skin | 1.361 | 0.001 |
| RT112/84 | null | bladder | 0.395 | 0.578 |
| AM-38 | null | brain | 0.499 | 6.774 |
| UM-UC-3 | null | bladder | 1.945 | 2.770 |
| CHL-1 | WT | skin | 1.539 | 2.663 |
| A-375 | WT | skin | 2.817 | 6.554 |
| 5637 | WT | bladder | 4.220 | 11.338 |
| TCCSUP | WT | bladder | 1.375 | 0.791 |
| T24 | WT | bladder | 5.506 | >20 |
| D283 MED | WT | brain | 8.196 | 19.702 |
| SK-MEL-3 | WT | skin | 9.811 | 10.232 |
| SK-MEL-5 | null | skin | 3.376 | 6.266 |
| G-361 | WT | skin | 14.766 | 0.102 |
| D341 Med | WT | brain | 11.843 | 0.731 |
| Hs 852.T | WT | skin | 17.235 | 19.244 |
| Malme-3M | null | skin | >20 | >20 |
| SK-MEL-24 | null | skin | >20 | 14.226 |
| Hs 934.T | WT | skin | 0.230 | 9.282 |
| A101D | null | skin | 0.109 | 0.088 |
| Reh | null | heme | 3.782 | 2.217 |
| WM-266-4 | WT | skin | 4.030 | 1.266 |
| A2058 | WT | skin | 2.030 | 6.732 |
| LN-229 | WT | brain | 1.353 | 1.128 |
| HT1376 | WT | bladder | 5.649 | >20 |
| NCI-H1568 | WT | lung | 4.306 | 16.885 |
| NCI-H929 | WT | heme | 5.965 | 23.811 |
| RPMI-8226 | WT | heme | 8.132 | 21.801 |
| SKM-1 | WT | heme | 1.746 | >20 |
| KALS-1 | WT | brain | 9.902 | >20 |
| COLO679 | WT | skin | 8.179 | 4.496 |
| RPMI-7951 | WT | skin | 1.169 | 11.916 |
| SK-MEL-1 | WT | skin | >20 | >20 |
| C32 | WT | skin | 1.906 | 2.259 |
| WM-115 | WT | skin | 1.783 | 6.376 |
| SK-MEL-28 | WT | skin | 1.257 | >20 |
| HEL9217 | null | heme | 0.229 | 0.202 |
| MEG-01 | WT | heme | 0.608 | 0.416 |
| REC1 | WT | heme | 0.799 | 0.985 |
| JM1 | null | heme | 0.990 | 0.763 |
| SUP-B15 | null | heme | 0.892 | 0.618 |
| HEL | null | heme | 1.006 | 1.844 |
| EB2 | WT | heme | 1.332 | 5.502 |
| KU812 | WT | heme | 2.655 | 5.283 |
| RL | WT | heme | 1.757 | 0.423 |
| NOMO-1 | null | heme | 5.071 | 0.269 |
| SH4 | null | skin | 1.917 | 1.195 |
| DB | WT | heme | 4.618 | 3.885 |
| RS4; 11 | null | heme | 4.478 | 1.488 |
| HL60 | WT | heme | 5.813 | 3.212 |
| BCP-1 | WT | heme | 6.508 | 0.107 |
| KASUMI1 | WT | heme | 8.943 | 5.404 |
| CA46 | WT | heme | 8.362 | 0.719 |
| RAJI | WT | heme | 8.362 | 0.788 |
| SK-MEL-31 | WT | skin | 10.649 | 2.041 |
| EB1 | WT | heme | 11.520 | 1.035 |
| NAMALWA | WT | heme | 19.282 | 0.000 |
| SK-MEL-24 | null | skin | 31.337 | 0.450 |
| HUT78 | null | heme | 0.503 | 0.299 |
| TOLEDO | WT | heme | 1.076 | 2.047 |
| Panc 03.27 | null | panc | 1.383 | 0.696 |
| HUT102 | WT | heme | 1.211 | 2.453 |
| SUDHL6 | WT | heme | 2.407 | 3.702 |
| LN-229 | WT | brain | 2.752 | 0.108 |
| Panc 10.05 | WT | panc | 2.844 | 1.328 |
| Daudi | WT | heme | 5.069 | 6.917 |
| SUPT1 | WT | heme | 4.657 | 7.947 |
| MOLT4 | WT | heme | 5.855 | 13.816 |
| U266B1 | WT | heme | 5.314 | 5.525 |
| Panc 02.03 | WT | panc | 6.640 | 2.656 |
| LOUCY | WT | heme | 9.192 | 6.823 |
| TALL1 | WT | heme | 8.538 | >20 |
| ST486 | WT | heme | 0.613 | 0.257 |
| HUP-T4 | null | panc | 0.700 | 0.220 |
| MIA PaCa-2 | null | panc | 0.517 | 1.246 |
| KP-4 | null | panc | 0.359 | 1.423 |
| HPAC | WT | panc | 1.294 | 4.959 |
| HUP-T3 | null | panc | 0.644 | 0.413 |
| THP-1 | null | heme | 1.398 | 0.513 |
| CFPAC-1 | WT | panc | 0.901 | 0.542 |
| PSN1 | null | panc | 2.946 | 4.313 |
| Panc 05.04 | WT | panc | 1.048 | 7.773 |
| BxPC-3 | null | panc | 1.695 | 2.601 |
| Panc 04.03 | WT | panc | 7.407 | 1.283 |
| PANC-1 | null | panc | 10.316 | 11.727 |
| HH | WT | heme | 13.138 | 0.018 |
| HT | WT | heme | 11.680 | 9.479 |
| MC116 | WT | heme | 16.238 | 1.662 |
| Mino | WT | heme | 14.508 | 8.845 |
| KASUMI6 | WT | heme | >20 | 0.142 |
| KG1 | WT | heme | 24.366 | 27.277 |
| U937 | WT | heme | 1.988 | 39.785 |
| JVM2 | WT | heme | 1.496 | 1.723 |
| P3HR-1 | WT | heme | 0.103 | 0.042 |
| Hs 766T | WT | panc | >20 | 0.576 |
| PL45 | WT | panc | >20 | >20 |
| SW 1990 | WT | panc | 54.601 | 0.743 |
| HPAF-II | WT | panc | 1.063 | 3.541 |
| KP-2 | WT | panc | 0.000 | 8.705 |
| AsPC-1 | WT | panc | 17.503 | 5.492 |
| F-36P | null | heme | 0.398 | 0.247 |
| Capan-1 | null | panc | 0.703 | 0.514 |
| Pfeiffer | WT | heme | 1.996 | 0.859 |
| GDM1 | WT | heme | 7.738 | 3.414 |
| D341 MED | WT | brain | 19.049 | 1.140 |
| QGP-1 | WT | panc | 43.654 | 1.567 |
| AML-193 | WT | heme | 1.447 | 8.164 |
| NALM-1 | null | heme | 3.424 | 2.871 |
| NCI-H69 | WT | lung | 1.046 | 0.379 |
| MM1S | WT | heme | 3.859 | 8.932 |
| NCI-H524 | WT | lung | 0.740 | 24.999 |
| NCI-H2228 | null | lung | 0.969 | 0.703 |
| HUP-T3 | null | panc | 0.501 | 0.599 |
| NCI-H647 | null | lung | 0.753 | 0.694 |
| MIA PaCa-2 | null | panc | 0.852 | 0.217 |
| NCI-H1755 | null | lung | 2.420 | 1.372 |
| NCI-H226 | WT | lung | 1.515 | 0.831 |
| NCI-H1975 | WT | lung | 1.515 | 0.831 |
| NCI-H1944 | WT | lung | 5.125 | 0.478 |
| NCI-H1915 | WT | lung | 7.065 | 5.852 |
| NCI-H1299 | WT | lung | 5.026 | >20 |
| Capan 2 | WT | panc | 7.248 | 6.810 |
| RERF-LC-Sq1 | null | lung | 5.454 | 0.493 |
| NCI-H292 | null | lung | 5.750 | 0.842 |
| Malme-3M | null | skin | 7.819 | 14.205 |
| LU-DLU-1 | null | lung | 2.145 | 0.658 |
| MDA-MB-361 | WT | breast | >20 | 5.643 |
| HCC202 | WT | breast | >20 | >20 |
| NCI-H196 | WT | lung | >20 | 0.626 |
| KP4 | null | panc | 0.251 | 1.831 |
| HCC15 | null | lung | 0.357 | 0.204 |
| SU.86.86 | null | panc | 0.935 | 0.845 |
| NCI-H1703 | WT | lung | 13.537 | 8.721 |
| MDA-MB-134-VI | WT | breast | 4.785 | >20 |
| HCC1937 | WT | breast | 1.060 | >20 |
| MJ | WT | heme | >20 | 0.026 |
| BDCM | WT | heme | 89.161 | 16.145 |
| NCI-H2030 | WT | lung | 2.199 | 1.268 |
| NCI-H1838 | WT | lung | 1.061 | 0.930 |
| NCI-H1563 | null | lung | 1.683 | 0.264 |
| MCF-7 | null | breast | 0.989 | 0.717 |
| HCC1395 | null | breast | 2.069 | 0.657 |
| HCC38 | WT | breast | 3.263 | 1.795 |
| MDA-MB-453 | WT | breast | 3.672 | 4.680 |
| HCC1954 | WT | breast | 4.128 | 8.622 |
| LK-2 | WT | lung | 6.260 | 1.775 |
| BT474 | WT | breast | 2.502 | 0.027 |
| Hs 578T | WT | breast | 6.956 | 9.053 |
| HCC1428 | WT | breast | 9.441 | 10.594 |
| NCI-H2172 | WT | lung | 8.970 | 0.989 |
| HCC70 | WT | breast | 7.105 | 2.298 |
| NCI-H146 | WT | lung | 1.566 | >20 |
| MDA-MB-175-VII | WT | breast | 11.262 | >20 |
| NCI-H522 | WT | lung | 13.416 | 18.612 |
| AU565 | WT | breast | 19.738 | 2.393 |
| DU4475 | WT | breast | >20 | >20 |
| HCC1143 | WT | breast | >20 | 8.529 |
| HCC1806 | WT | breast | >20 | 0.457 |
| T-47D | WT | breast | >20 | 4.795 |
| CAMA-1 | WT | breast | 25.406 | 1.133 |
| NCI-H520 | WT | lung | 27.989 | 2.142 |
| NCI-H2291 | WT | lung | 4.790 | 0.420 |
| HCC44 | WT | lung | 0.692 | 1.385 |
| MDA-MB-436 | WT | breast | 2.248 | 5.193 |
| NCI-H441 | WT | lung | 0.694 | 0.746 |
| HCC1419 | WT | breast | 2.222 | 0.146 |
| NCI-H2347 | WT | lung | 0.442 | 0.187 |
| NCI-H1930 | WT | lung | 8.053 | 6.523 |
| BT-549 | WT | breast | 7.090 | 0.445 |
| NCI-H1693 | WT | lung | 9.512 | >20 |
| BT-20 | WT | breast | 18.422 | >20 |
| HCC2157 | WT | breast | >20 | 3.620 |
| MDA-MB-415 | WT | breast | 19.726 | >20 |
| UACC-812 | WT | breast | 35.105 | 0.683 |
| Hs 739.T | WT | breast | 1.222 | 191.391 |
| NCI-H889 | WT | lung | 23.187 | 11.735 |
| NCI-H1703 | WT | lung | 23.738 | 8.911 |
| NCI-H2444 | WT | lung | 33.212 | 6.953 |
| NCI-H2170 | null | lung | 0.824 | 0.700 |
| HCT116 MTAP−/− | null | colon | 0.659 | 0.277 |
| HCT116 MTAP wt | WT | colon | 5.207 | 15.323 |
| HCC1187 | WT | breast | 0.582 | 1.700 |
| Hs 606.T | WT | breast | 1.340 | 1.162 |
| NCI-H446 | WT | lung | 2.015 | 2.405 |
| NCI-H2122 | WT | lung | 5.229 | 0.001 |
| HCC1599 | WT | breast | 6.037 | 3.249 |
| NCI-H526 | WT | lung | 11.991 | 0.263 |
| HCC2218 | WT | breast | 13.118 | >20 |
| NCI-H358 | WT | lung | 1.428 | 0.403 |
| NCI-H23 | WT | lung | 3.565 | 21.483 |
| NCI-H82 | WT | lung | 4.760 | 33.337 |
| HCC366 | WT | lung | 8.414 | 70.746 |
| SK-BR-3 | WT | breast | 9.993 | 16.009 |
| UACC-893 | WT | breast | 15.015 | 33.788 |
| MDA-MB-157 | WT | breast | 19.566 | >20 |
| OE33 | WT | esophageal | 0.921 | 0.798 |
| TE-10 | null | esophageal | 0.843 | 0.882 |
| TE-15 | null | esophageal | 1.657 | 1.698 |
| GRANTA-519 | null | heme | 2.036 | 0.421 |
| TE-14 | null | esophageal | 1.994 | 3.952 |
| OE19 | WT | esophageal | 3.880 | 16.026 |
| OCI-LY19 | null | heme | 5.902 | 7.667 |
| SU-DHL-4 | WT | heme | 7.434 | 13.328 |
| KYSE-510 | WT | esophageal | 9.418 | >20 |
| KYSE-180 | WT | esophageal | 9.858 | 10.349 |
| HCC1569 | WT | breast | >20 | >20 |
| MM.1S | WT | heme | 10.955 | 16.561 |
| HCT116 MTAP−/− | null | colon | | |
| T.T | WT | esophageal | 3.115 | 8.420 |
| EC-GI-10 | WT | esophageal | 39.047 | 5.632 |
| TE-4 | WT | esophageal | 10.018 | 15.434 |
| TE-11 | WT | esophageal | 7.007 | 1.584 |
| TF-1 | WT | heme | 2.559 | 1.371 |
| TE-9 | WT | esophageal | 5.632 | 25.186 |
| OPM-2 | WT | heme | 8.483 | 0.029 |
| OCI-AML-2 | WT | heme | 2.259 | 18.758 |
| KYSE-140 | WT | esophageal | 2.953 | 1.875 |
| HCT116 MTAP wt | WT | colon | | |
| Z-138 | WT | heme | 24.954 | 20.204 |
| DOHH-2 | null | heme | 1.120 | 0.495 |
| ZR-75-30 | WT | breast | 50.312 | 33.299 |
| ZR-75-1 | WT | breast | 19.106 | 22.777 |
| TE-8 | WT | esophageal | 7.429 | 38.030 |
| Kopn-8 | WT | heme | 6.946 | 13.265 |
| OCI-M1 | WT | heme | 5.633 | 35.024 |
| TE-5 | WT | esophageal | 15.301 | 69.747 |
| KYSE-150 | WT | esophageal | 22.057 | 25.111 |
| TE-6 | null | esophageal | 19.731 | 1.026 |
| LP-1 | WT | heme | 2.396 | 0.687 |
| WSU-DLCL2 | WT | heme | 5.645 | 13.844 |
| KMS-12-BM | WT | heme | 7.360 | 30.068 |
| U-937 | WT | heme | 1.122 | 1.773 |
| KG-1 | WT | heme | 2.214 | 13.265 |
| CCRF-CEM | null | heme | 3.489 | 12.392 |
| AML-193 | WT | heme | 15.814 | 20.501 |
| OE21 | WT | esophageal | 1.230 | 1.615 |
| KYSE-270 | WT | esophageal | 1.611 | 1.411 |
| TE-1 | WT | esophageal | 2.307 | 4.424 |
| KYSE-70 | WT | esophageal | 4.864 | 2.456 |
| KYSE-30 | WT | esophageal | 6.490 | 4.350 |
| KYSE-410 | WT | esophageal | 5.424 | 3.269 |
| OCI-AML-5 | WT | heme | 4.838 | 29.590 |
| OCI-AML-3 | WT | heme | 11.460 | >20 |

The data shown in table 1 demonstrate that tumor cells that are MTAP null, grown either in cell culture or in vivo, show unexpected sensitivity to inhibition by MAT2A inhibitors. The data indicates that the MTAP status determines the level of sensitivity of tumors to MAT2A inhibitors. It is demonstrated that the level of sensitivity of tumors to MAT2A inhibitors can be assessed by determining the status of MTAP expressed by a tumor cell. For example, tumor cells in which the MTAP gene is not present (i.e. MTAP null) or expression is downregulated or MTAP protein function is impaired, correlates with higher sensitivity to MAT2A inhibitors than tumor cells having normal MTAP gene expression and MTAP protein function. Thus, these observations can form the basis of valuable new diagnostic methods for predicting the effects of MAT2A inhibitors on tumor growth, and give oncologists an additional tool to assist them in choosing the most appropriate treatment for their patients.

Accordingly, the present invention provides a method for treating a cancer in a subject wherein said tumor is characterized by reduction or absence of MTAP expression or absence of the MTAP gene or reduced function or nonfunction of MTAP protein said method comprising administering to the subject a therapeutically effective amount of a MAT2A inhibitor. In an embodiment, the cancer is characterized by the absence of MTAP i.e. it is MTAP null. In another embodiment, the cancer is characterized by reduced expression of the MTAP gene, for example, to the extent that the level of MTA in the cancer is sufficient to inhibit PRMT5 methylation activity. In another embodiment, the cancer is characterized by reduced function or nonfunction of MTAP protein, for example, to the extent that the level of MTA in the cancer is elevated to an extent that inhibits normal PRMT5 methylation activity. PRMT5 inhibitor include, without limitation, those described in WO/2014/145214, WO/2014/100716, WO/2014/100730, WO/2014/100695, WO/2014/100734 and WO/2011/079236.

In a particular embodiment, the invention provides a method of treating an MTAP null cancer in a subject comprising administering to the subject a therapeutically effective amount of a MAT2A inhibitor. In an embodiment, the foregoing method further comprises detecting the absence of the MTAP gene in the cancer, e.g. from a sample of the cancer taken from the patient.

“Cancer” in a mammal refers to the presence of cells possessing characteristics typical of cancers, such as uncontrolled proliferation, immortality, metastatic potential, rapid growth and proliferation rate, and certain characteristic morphological features. The term cancer and tumor is used herein interchangeably. Often, cancer cells will be in the form of a solid tumor, but such cells may exist alone within an animal, or may circulate in the blood stream as independent cells, such as leukemic cells.

The term “treating” as used herein, unless otherwise indicated, means reversing, alleviating, inhibiting the progress of, or preventing, either partially or completely, the growth of tumors, tumor metastases, or other cancer-causing or neoplastic cells in a patient. The term “treatment” as used herein, unless otherwise indicated, refers to the act of treating. A “method of treating cancer” refers to a procedure or course of action that is designed to reduce or eliminate the number of cancer cells in an animal, or to alleviate the symptoms of a cancer.

The term “effective amount” or “effective amount” means the amount of the MAT2A inhibitor compound or combination with another drug that will elicit the biological or medical response of a tissue, system or animal e.g. human that is being sought. In an embodiment, the response is inhibition of tumor volume or the rate of increase in tumor volume over time, for example, static volume or decreased volume. In another embodiment, an effective amount is the amount of MAT2A inhibitor that reduces the number of cancer cells or the reduces the rate of increase in number of cancer cells. In another embodiment, an effective amount is the amount of MAT2A inhibitor sufficient to cause differentiation of at least a portion of the cancer cells, for example, in hematological tumors the conversion of undifferentiated blast cells to functional neutrophils. A therapeutically effective amount does not necessarily mean that the cancer cells will be entirely eliminated or that the number of cells will be reduced to zero or undetectable, or that the symptoms of the cancer will completely alleviated.

Expression level and the presence or absence of the MTAP gene and the function of MTAP protein in a tumor or tumor cell may be determined using standard techniques. For example, methods for determining MTAP status in tumor cells is described in U.S. Pat. No. 5,942,393 using oligonucleotide probes. Norbori et al. ((1991) Cancer Res. 51:3193-3197); and (1993) Cancer Res. 53:1098-1101) describe the use of a polyclonal antisera to bovine MTAP to detect MTAP protein isolated from tumor cell lines or primary tumor specimens in an immunoblot analysis. Garcia-Castellano et al. (2002, supra) describe the use of antihuman MTAP chicken antibody to screen osteosarcoma tumor samples that were embedded in OCT frozen blocks. MTAP protein function can be determined by sequencing the MTAP protein to identify any loss-of-function mutations or else isolating the protein from a sample and measuring its ability to convert MTA into methionine and/or adenine either directly or indirectly.

In another aspect of the invention, there is provided a method for inhibiting proliferation or survival of a cancer cell wherein said cancer cell is characterized by reduction or absence MTAP expression or absence of the MTAP gene or reduced function of MTAP protein said method comprising contacting said cancer cell with an effective amount of a MAT2A inhibitor.

In another aspect, the present invention provides a method of diagnosing a tumor in a patient comprising determining in a sample of said tumor reduced level of an MTAP gene expression, the absence of an MTAP gene or reduction of the level or function of MTAP protein and administering to said patient a therapeutically acceptable amount of a MAT2A inhibitor.

In another aspect, the present invention provides a method for characterizing a tumor cell comprising measuring in said tumor cell the level of MTAP gene expression, the presence or absence of an MTAP gene or the level of MTAP protein present, wherein the reduction or absence MTAP expression or absence of the MTAP gene or reduced level or function of MTAP protein relative to a reference cell indicates that survival or proliferation of said tumor cell can be inhibited by a MAT2A inhibitor.

In another aspect of the present invention, there is provided a method for determining whether survival or proliferation of a tumor cell can be inhibited by contacting said tumor cell with a MAT2A inhibitor, said method comprising determining the status of MTAP in said tumor cell, wherein the reduction or absence MTAP expression or absence of the MTAP gene or reduced level or function of MTAP protein indicates survival or proliferation of said tumor cell can be inhibited by a MAT2A inhibitor.

Further genomic analysis of cell lines in table 1 revealed that in 16 MTAP null cell lines that also incorporate a KRAS mutation 14 (88%) were sensitive to MAT2A inhibition with AGI-512 and AGI-673 compared to 24 of 49 (49%) of MTAP wild type cell lines sensitive when a KRAS mutation was present (p.008). Furthermore, it was discovered that the presence of a co-mutation p53 mutation with MTAP null status correlated with improved sensitivity to MAT2A inhibitors compared to the absence of a p53 mutation. See table 2.

TABLE 2

| | | MTAP | KRAS | P53 | AGI-673 | AGI-512 |
|---|---|---|---|---|---|---|
| CELL LINE | TISSUE | STATUS | STATUS | STATUS | IC50 (uM) | IC50 (uM) |
| JM1 | heme | null | WT | WT | 1.028 | 0.756 |
| HUT78 | heme | null | WT | p.R196* | 0.602 | 0.395 |
| LN-18 | brain | null | WT | p.C238S | 0.429 | 0.616 |
| SW 1088 | brain | null | WT | p.R273C | 0.876 | 0.905 |
| HCC15 | lung | null | WT | p.D259V | 0.545 | 0.334 |
| Granta-519 | heme | null | WT | WT | 2.609 | 1.093 |
| DOHH-2 | heme | null | WT | WT | 1.501 | 0.627 |
| F-36P | heme | null | WT | p.Y126_splice | 0.513 | 0.338 |
| HUP-T4 | pancreas | null | p.G12V | p.I255T | 0.960 | 1.185 |
| HUP-T3 | pancreas | null | p.G12R | p.R282W | 1.034 | 5.849 |
| MIA PaCa-2 | pancreas | null | p.G12C | p.R248W | 0.984 | 2.707 |
| Panc 03.27 | pancreas | null | p.G12V | WT | 1.503 | 4.937 |
| PSN1 | pancreas | null | p.G12R | p.K132Q | 2.917 | 8.675 |
| Capan-1 | pancreas | null | p.G12V | p.A159V | 1.788 | 3.131 |
| MDA-MB-231 | breast | null | p.G13D | p.R280K | 0.842 | 1.210 |
| NCI-H647 | lung | null | p.G13D | p.S261_splice | 1.116 | 1.947 |
| SU.86.86 | pancreas | null | p.G12D | p.G245S | 1.230 | 2.398 |
| UM-UC-3 | bladder | null | p.G12C | WT | 1.915 | 4.249 |
| A101D | skin | null | WT | WT | 0.157 | 0.159 |
| AM-38 | brain | null | WT | WT | 1.169 | 9.518 |
| H4 | brain | null | WT | WT | 0.788 | 1.175 |
| HEL | heme | null | WT | p.M133K | 1.133 | 2.817 |
| IGR-1 | skin | null | WT | WT | 0.050 | 0.025 |
| GB-1 | brain | null | WT | WT | 2.004 | 3.697 |
| KS-1 | brain | null | WT | WT | 2.134 | 20.000 |
| HCC1395 | breast | null | WT | p.R175H | 4.180 | 4.011 |
| K-562 | heme | null | WT | p.Q136fs | 0.764 | 1.510 |
| MCF-7 | breast | null | WT | WT | 1.418 | 4.318 |
| NCI-H1437 | lung | null | WT | p.R267P | 2.020 | 1.944 |
| RT-112 | bladder | null | WT | p.R248Q | 0.765 | 1.095 |
| RT112/84 | bladder | null | WT | p.R175H | 0.453 | 0.694 |
| SW 780 | bladder | null | WT | NA | 1.098 | 1.850 |
| TE-10 | esophageal | null | WT | p.C242Y | 1.781 | 1.858 |
| THP-1 | heme | null | WT | p.R174fs | 1.882 | 3.825 |

*N-terminal fragment 1-195

Accordingly, the methods of the invention further provide determining the presence of a mutant KRAS or p53 in the cancer or a cancer cell whereby the presence of a KRAS or p53 mutation indicates the cancer or cancer cell is susceptible to treatment with a MAT2A inhibitor. By mutant KRAS, or KRAS mutation, is meant KRAS protein incorporating an activating mutation that alters its normal function and the gene encoding such a protein. For example, a mutant KRAS protein may incorporate a single amino acid substitution at position 12 or 13. In a particular embodiment, the KRAS mutant incorporates a G12X or G13X substitution. In a particular embodiment, the substitution is G12V, G12R, G12C or G13D. In another embodiment, the substitution is G13D. By “mutant p53” or “p53 mutation” is meant p53 protein (or gene encoding said protein) incorporating a mutation that inhibits or eliminates its tumor suppressor function. Examples of p53 mutations applicable to the invention are shown in table 2.

Accordingly, the present invention provides a method for treating a cancer in a subject wherein said cancer is characterized by reduction or absence MTAP expression or absence of the MTAP gene or reduced function of MTAP protein said method comprising administering to the subject a therapeutically effective amount of a MAT2A inhibitor wherein said cancer is further characterized by the presence of mutant KRAS or mutant p53.

The present invention provides a method for determining whether survival or proliferation of a tumor cell can be inhibited by contacting said tumor cell with a MAT2A inhibitor, said method comprising determining the status of MTAP and the presence of a KRAS or p53 mutation in said tumor cell, wherein the reduction or absence MTAP expression or absence of the MTAP gene or reduced level or function of MTAP protein in addition to a KRAS or p53 mutation indicates survival or proliferation of said tumor cell can be inhibited by a MAT2A inhibitor.

In another aspect, the present invention provides a method for characterizing a tumor cell comprising measuring in said tumor cell the level of MTAP gene expression, the presence or absence of an MTAP gene or the level of MTAP protein present and determining the presence of a KRAS or p53 mutation, wherein the reduction or absence MTAP expression or absence of the MTAP gene or reduced level or function of MTAP protein relative to a reference cell and the presence of a KRAS or p53 mutation indicates that survival or proliferation of said tumor cell can be inhibited by a MAT2A inhibitor.

In another aspect, the present invention provides a method of determining the responsiveness of a tumor to MAT2A inhibition comprising determining in a sample of said tumor a reduced expression level of an MTAP gene, the absence of an MTAP gene or reduction of the level or function of MTAP protein in combination with a KRAS or p53 mutation, wherein a reduced expression level of an MTAP gene, the absence of an MTAP gene or reduction of the level or function of MTAP protein and the presence of a KRAS or p53 mutation indicates said tumor is responsive to a MAT2A inhibitor.

In another aspect, the present invention provides a kit comprising a reagent for measuring in a tumor sample the expression level of an MTAP gene, the absence of an MTAP gene or reduction of the level or function of MTAP protein and the presence of a KRAS or p53 mutation, said kit further comprising instructions for administering a therapeutically effective amount of a MAT2A inhibitor.

In the methods described herein the tumor cell will typically be from a patient diagnosed with cancer, a precancerous condition, or another form of abnormal cell growth, and in need of treatment. The cancer may be lung cancer (e.g. non-small cell lung cancer (NSCLC)), pancreatic cancer, head and neck cancer, gastric cancer, breast cancer, colon cancer, ovarian cancer, or any of a variety of other cancers described herein below.

In the methods of this invention, MTAP expression level and MTAP protein function can be assessed relative to that in a reference cell, e.g. a non-cancerous cell. In the methods of this invention, the level of MTAP expressed by a tumor cell can be assessed by using any of the standard bioassay procedures known in the art for determination of the level of expression of a gene, including for example ELISA, RIA, immunoprecipitation, immunoblotting, immunofluorescence microscopy, RT-PCR, in situ hybridization, cDNA microarray, or the like, as described in more detail below. In the methods of this invention, the expression level of MTAP is preferably assessed by assaying a biopsy.

In the methods of this invention, the cancer cell can be any tissue type, for example, pancreatic, lung, bladder, breast, esophageal, colon, ovarian. In a particular embodiment, the cancer cell is pancreatic. In another embodiment, the cancer cell is lung. In another embodiment, the cancer cell is esophageal. The tumor cell is preferably of a type known to or expected to be MTAP null.

MAT2A inhibitors are any agent that modulates MAT2A function, for example, an agent that interacts with MAT2A to inhibit or enhance MAT2A activity or otherwise affect normal MAT2A function. MAT2A function can be affected at any level, including transcription, protein expression, protein localization, and cellular or extra-cellular activity. In the methods of this invention, the MAT2A inhibitor can be any MAT2A inhibitor. In a particular embodiment, the MAT2A inhibitor is an oligonucleotide that represses MAT2A gene expression or product activity by, for example, binding to and inhibiting MAT2A nucleic acid (i.e. DNA or mRNA). In a particular embodiment, the MAT2A inhibitor is an oligonucleotide e.g. an antisense oligonucleotide, shRNA, siRNA, microRNA or an aptamer. In a particular embodiment, the MAT2A inhibitor is a oligonucleotide, for example, as described in WO2004065542. In a particular embodiment, the MAT2A inhibitor is an siRNA, for example, as described in patent application CN 2015-10476981 or in Wang et al, Zhonghua Shiyan Waike Zazhi, 2009, 26(2):184-186 or Wang et al, Journal of Experimental & Clinical Cancer Research (2008) volume 27. In a particular embodiment, the MAT2A inhibitor is a microRNA oligonucleotide, for example, as described in US patent application publication no. 20150225719 or in Lo et al, PLoS One (2013), 8(9), e75628. In an embodiment, the MAT2A inhibitor is an antibody that binds to MAT2A.

In a particular embodiment, the MAT2A inhibitor is a small molecule compound, e.g. AGI-512 or AGI-673. In an embodiment, the MAT2A inhibitor is a fluorinated N,N-dialkylaminostilbene described in Zhang et al, ACS Chem Biol, 2013, 8(4):796-803. In an embodiment, the MAT2A inhibitor is a 2′,6′-dihalostyryaniline, pyridine or pyrimidine described in Sviripa et al, J Med Chem, 2014, 57:6083-6091 In a particular embodiment the compound is selected from the group consisting of compound 1a-12b:

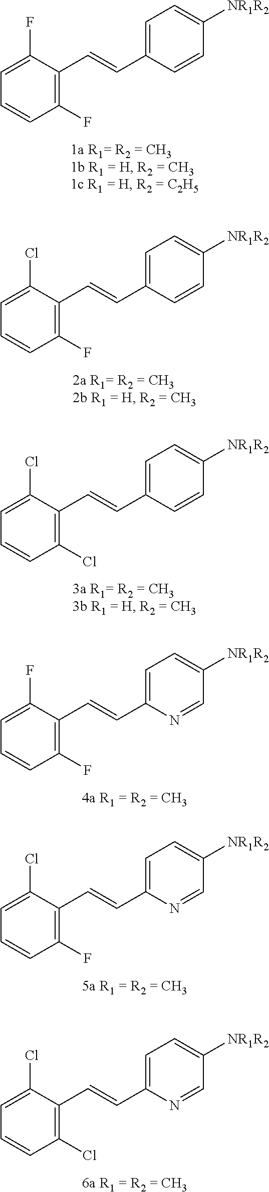

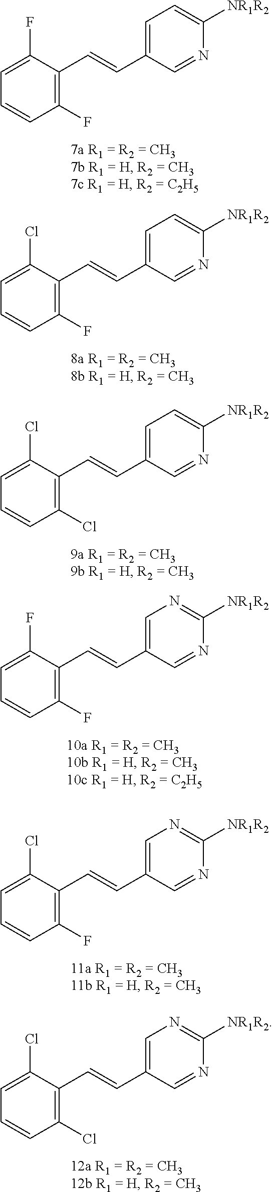

In another embodiment, the MAT2A inhibitor is a compound disclosed in WO2012103457. In an embodiment, the MAT2A inhibitor is a compound of the formula:

X—Ar₁—CR^{a}═CR^{b}—Ar₂

where R^{a} and R^{b} are independently H, alkyl, halo, alkoxy, cyano; X represents at least one halogen, e.g., a fluorine, chlorine, bromine, or iodine substituent, on Art; each of Ar₁ and Ar₂ are aryl, e.g., phenyl, naphthyl, and heteroaryl e.g., pyridyl, pyrolidyl, piperidyl, pyrimidyl, indolyl, thienyl, which can be further substituted with halo, amino, alkylamino, dialkylamino, arylalkylamino, N-oxides of dialkylamino, trialkylammonium, mercapto, alkylthio, alkanoyl, nitro, nitrosyl, cyano, alkoxy, alkenyloxy, aryl, heteroaryl, sulfonyl, sulfonamide, CONR₁₁R₁₂, NR₁₁CO(R₁₃), NR₁₁COO(R₁₃), NR₁₁CONR₁₂Rₙ where R₁₁, R₁₂, R₁₃ are independently, H, alkyl, aryl, heteroaryl or a fluorine; provided that Ar₂ contains at least one nitrogen atom in the aryl ring or at least one nitrogen substituent on the aryl ring e.g., an NR^{c}R^{d}Z substituent on Ar₂ where R^{c} is H, alkyl, alkoxy, aryl, heteroaryl, R^{d} is an alkyl group, Z is a an unshared pair of electrons, H, alkyl, oxygen.

In another embodiment, the MAT2A inhibitor is a compound of formula:

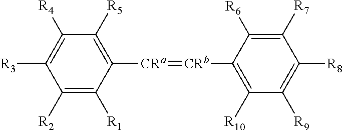

where R^{a} and R^{b} are as defined above, R₁ to R₁₀ are independently H, halo, amino, alkylamino, dialkylamino, N-oxides of dialkylamino, aralkylamino, dialkyloxyamino, trialkylammonium, mercapto, alkylthio, alkanoyl, nitro, nitrosyl, cyano, alkoxy, alkenyloxy, aryl, heteroaryl, sulfonyl, sulfonamide, CONR₁₁R₁₂, NR₁₁CO(R₁₃), NR₁₁COO(R₁₃), NR₁₁CONR₁₂R₁₃ where R₁₁, R₁₂, R₁₃, are independently, H, alkyl, aryl, heteroaryl or a fluorine; provided at least one of R₁ to R₅ is a halogen, e.g. a fluorine and/or chlorine; and at least one of R₆ to R₁₀ is a nitrogen containing substituent, e.g., an NR^{c}R^{d}Z substituent where R^{c} is H, alkyl, e.g., a lower alkyl, alkoxy, aryl, heteroaryl, R^{d} is an alkyl group, Z is a an unshared pair of electrons, H, alkyl, oxygen, or a pharmaceutically acceptable salt thereof or a biotinylated derivative thereof.

In another embodiment, the MAT2A inhibitor is a compound of formula:

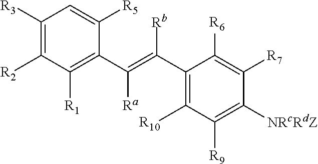

where R₁, R₂, R₃, R₅, R₆, R₇, R₉, R₁₀, R^{a}, R^{b} and NR^{c}R^{d}Z are the same as defined above, or pharmaceutically acceptable salts thereof or a biotinylated derivative thereof. In one aspect of the present disclosure, R^{a}, R^{b} are both H, one or more of R₁, R₂, R₃, or R₅, are fluorine or chlorine and R^{c} is H or lower alkyl, such as a methyl, ethyl, propyl group, and R^{d} is a lower alkyl, such as a methyl, ethyl, propyl group. In an embodiment, the MAT2A inhibitor is selected from the group consisting of: (E)-4-(2-Fluorostyryl)-N,N-dimethylaniline; (E)-4-(3-Fluorostyryl)-N,N-dimethylaniline; (E)-4-(4-Fluorostyryl)-N,N-dimethylaniline; (E)-4-(2-Fluorostyryl)-N,N-diethyl aniline; (E)-4-(2-Fluorostyryl)-N,N-diphenylaniline; (E)-1-(4-(2-Fluorostyryl)phenyl)-4-methylpiperazine; (E)-4-(2-Fluorostyryl)-N,N-dimethylnaphthalen-1-amine; (E)-2-(4-(2-Fluorostyryl)phenyl)-1-methyl-1H-imidazole; (E)-4-(2,3-Difluorostyryl)-N,N-dimethylaniline; (E)-4-(2,4-Difluorostyryl)-N,N-dimethylaniline; (E)-4-(2,5-Difluorostyryl)-N,N-dimethylaniline; (E)-2-(2,6-Difluorostyryl)-N,N-dimethylaniline; (E)-3-(2,6-Difluorostyryl)-N,N-dimethylaniline; (E)-4-(2,6-Difluorostyryl)-N,N-dimethylaniline; (E)-4-(2,6-Difluorostyryl)-N,N-diethylaniline; (E)-4-(3,4-Difluorostyryl)-N,N-dimethylaniline; (E)-4-(3,5-Difluorostyryl)N, N-dimethylaniline; (E)-N,N-Dimethyl-4-(2,3,6-trifluorostyryl)aniline; (E)-N,N-Dimethyl-4-(2,4,6-trifluorostyryl)aniline; (E)-4-(2-chloro-6-fluorostyryl)-N,N-dimethylaniline; (E)-4-(2,6-di chlorostyryl)-N,N-dimethylaniline; (E)-4-(2,6-Difluorophenethyl)-N,N-dimethylaniline; and (E)-2-benzamide-4-(2,6-difluorostyryl)-N,N-dimethylaniline.

In another aspect of the invention, provided is a method for treating a cancer in a subject wherein said tumor is characterized by reduction or absence of MTAP expression or absence of the MTAP gene or reduced function or non function of MTAP protein said method comprising administering to the subject a therapeutically effective amount of a RIOK1 inhibitor. In an embodiment, a MAT2A inhibitor is co-administered with the RIOK1 inhibitor. In an embodiment, the cancer is characterized by the absence of MTAP i.e. it is MTAP null. In another embodiment, the cancer is characterized by reduced expression of the MTAP gene. In another embodiment, the cancer is further characterized by the presence of a KRAS or p53 mutation. In another aspect, there is provided a method for treating an MTAP null cancer comprising administering an effective amount of a RIOK1 inhibitor. In an embodiment, the cancer incorporates mutant KRAS or mutant p53.

In another aspect of the invention, provided is a method for treating a cancer in a subject wherein said tumor is characterized by reduction or absence of MTAP expression or absence of the MTAP gene or reduced function or nonfunction of MTAP protein said method comprising administering to the subject a therapeutically effective amount of a PRMT5 inhibitor. In an embodiment, a MAT2A inhibitor is co-administered with the PRMT5 inhibitor. In an embodiment, the cancer is characterized by the absence of MTAP i.e. it is MTAP null. In another embodiment, the cancer is characterized by reduced expression of the MTAP gene. In another embodiment, the cancer is further characterized by the presence of a KRAS or p53 mutation. In another aspect, there is provided a method for treating an MTAP null cancer comprising administering an effective amount of a PRMT5 inhibitor. In an embodiment, the cancer incorporates mutant KRAS or mutant p53.

In any of the above methods referring to a patient sample, an example of such a sample can be a tumor biopsy. For assessment of tumor cell MTAP expression, patient samples containing tumor cells, or proteins or nucleic acids produced by these tumor cells, may be used in the methods of the present invention. In these embodiments, the level of expression of MTAP can be assessed by assessing the amount (e.g. absolute amount or concentration) of MTAP in a tumor cell sample, e.g., a tumor biopsy obtained from a patient, or other patient sample containing material derived from the tumor (e.g. blood, serum, urine, or other bodily fluids or excretions as described herein above). The cell sample can, of course, be subjected to a variety of well-known post-collection preparative and storage techniques (e.g., nucleic acid and/or protein extraction, fixation, storage, freezing, ultrafiltration, concentration, evaporation, centrifugation, etc.) prior to assessing the amount of the marker in the sample. Likewise, tumor biopsies may also be subjected to post-collection preparative and storage techniques, e.g., fixation.

In another embodiment, expression of MTAP is assessed by preparing mRNA/cDNA (i.e. a transcribed polynucleotide) from cells or a in a patient sample, and by hybridizing the mRNA/cDNA with a reference polynucleotide which is a complement of MTAP nucleic acid, or a fragment thereof cDNA can, optionally, be amplified using any of a variety of polymerase chain reaction methods prior to hybridization with the reference polynucleotide. Expression of one or more biomarkers can likewise be detected using quantitative PCR to assess the level of expression of the MTAP.

The level of expression of MTAP in normal (i.e. non-cancerous) human tissue can be assessed in a variety of ways. In one embodiment, this normal level of expression is assessed by assessing the level of expression of the biomarker in a portion of cells which appears to be non-cancerous, and then comparing this normal level of expression with the level of expression in a portion of the tumor cells. Alternately, and particularly as further information becomes available as a result of routine performance of the methods described herein, population-average values for normal expression of the biomarkers of the invention may be used. In other embodiments, the ‘normal’ level of expression MTAP may be determined by assessing expression in a patient sample obtained from a non-cancer-afflicted patient, from a patient sample obtained from a patient before the suspected onset of cancer in the patient, from archived patient samples, and the like.

An exemplary method for detecting the presence or absence of MTAP protein or nucleic acid in a biological sample involves obtaining a biological sample (e.g. a tumor-associated body fluid) from a test subject and contacting the biological sample with a compound or an agent capable of detecting the polypeptide or nucleic acid (e.g., mRNA, genomic DNA, or cDNA). The detection methods of the invention can thus be used to detect mRNA, protein, cDNA, or genomic DNA, for example, in a biological sample in vitro as well as in vivo. For example, in vitro techniques for detection of mRNA include Northern hybridizations and in situ hybridizations. In vitro techniques for detection of a biomarker protein include enzyme linked immunosorbent assays (ELISAs), Western blots, immunoprecipitations and immunofluorescence. In vitro techniques for detection of genomic DNA include Southern hybridizations. In vivo techniques for detection of mRNA include polymerase chain reaction (PCR), Northern hybridizations and in situ hybridizations. Furthermore, in vivo techniques for detection of a biomarker protein include introducing into a subject a labeled antibody directed against the protein or fragment thereof. For example, the antibody can be labeled with a radioactive marker whose presence and location in a subject can be detected by standard imaging techniques.

A general principle of such diagnostic and prognostic assays involves preparing a sample or reaction mixture that may contain MTAP gene, and a probe, under appropriate conditions and for a time sufficient to allow the MAP gene and probe to interact and bind, thus forming a complex that can be removed and/or detected in the reaction mixture. These assays can be conducted in a variety of ways. For example, one method to conduct such an assay would involve anchoring the MTAP gene or fragment thereof or probe onto a solid phase support, also referred to as a substrate, and detecting target MTAP gene/probe complexes anchored on the solid phase at the end of the reaction. In one embodiment of such a method, a sample from a subject, which is to be assayed for presence and/or concentration of MTAP gene, can be anchored onto a carrier or solid phase support. In another embodiment, the reverse situation is possible, in which the probe can be anchored to a solid phase and a sample from a subject can be allowed to react as an unanchored component of the assay.

There are many established methods for anchoring assay components to a solid phase. These include, without limitation, MTAP gene or fragment thereof or probe molecules which are immobilized through conjugation of biotin and streptavidin. Such biotinylated assay components can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the surfaces with immobilized assay components can be prepared in advance and stored. Well-known supports or carriers include, but are not limited to, glass, polystyrene, nylon, polypropylene, nylon, polyethylene, dextran, amylases, natural and modified celluloses, polyacrylamides, gabbros, and magnetite.

In order to conduct assays with the above mentioned approaches, the non-immobilized component is added to the solid phase upon which the second component is anchored. After the reaction is complete, uncomplexed components may be removed (e.g., by washing) under conditions such that any complexes formed will remain immobilized upon the solid phase. The detection of MTAP gene/probe complexes anchored to the solid phase can be accomplished in a number of methods outlined herein. In one embodiment, the probe, when it is the unanchored assay component, can be labeled for the purpose of detection and readout of the assay, either directly or indirectly, with detectable labels discussed herein and which are well-known to one skilled in the art. It is also possible to directly detect MTAP gene/probe complex formation without further manipulation or labeling of either component (gene or probe), for example by utilizing the technique of fluorescence resonance energy transfer (i.e. FRET, see for example, Lakowicz et al., U.S. Pat. No. 5,631,169; Stavrianopoulos, et al., U.S. Pat. No. 4,868,103). A fluorophore label on the first, ‘donor’ molecule is selected such that, upon excitation with incident light of appropriate wavelength, its emitted fluorescent energy will be absorbed by a fluorescent label on a second ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘ acceptor’ molecule label in the assay should be maximal. A FRET binding event can be conveniently measured through standard fluorometric detection means well known in the art (e.g., using a fluorimeter).

In another embodiment, determination of the ability of a probe to recognize a biomarker can be accomplished without labeling either assay component (probe or MTAP gene) by utilizing a technology such as real-time Biomolecular Interaction Analysis (BIA) (see, e.g., Sjolander, S. and Urbaniczky, C., 1991, Anal. Chem. 63:2338-2345 and Szabo et al., 1995, Curr. Opin. Struct. Biol. 5:699-705). As used herein, “BIA” or “surface plasmon resonance” is a technology for studying biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

Alternatively, in another embodiment, analogous diagnostic and prognostic assays can be conducted with MTAP gene and probe as solutes in a liquid phase. In such an assay, the complexed biomarker and probe are separated from uncomplexed components by any of a number of standard techniques, including but not limited to: differential centrifugation, chromatography, electrophoresis and immunoprecipitation. In differential centrifugation, MTAP gene/probe complexes may be separated from uncomplexed assay components through a series of centrifugal steps, due to the different sedimentation equilibria of complexes based on their different sizes and densities (see, for example, Rivas, G., and Minton, A. P., 1993, Trends Biochem Sci. 18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the MTAP gene/probe complex as compared to the uncomplexed components may be exploited to differentiate the complex from uncomplexed components, for example through the utilization of ion-exchange chromatography resins. Such resins and chromatographic techniques are well known to one skilled in the art (see, e.g., Heegaard, N. H., 1998, J. Mol. Recognit. Winter 11(1-6):141-8: Hage, D. S., and Tweed, S. A. J. Chromatogr B Biomed Sci Appl 1997 Oct. 10; 699(1-2):499-525). Gel electrophoresis may also be employed to separate complexed assay components from unbound components (see, e.g., Ausubel et al., ed., Current Protocols in Molecular Biology, John Wiley & Sons, New York, 1987-1999). In this technique, protein or nucleic acid complexes are separated based on size or charge, for example. In order to maintain the binding interaction during the electrophoretic process, non-denaturing gel matrix materials and conditions in the absence of reducing agent are typically preferred. Appropriate conditions to the particular assay and components thereof will be well known to one skilled in the art.

In a particular embodiment, the level of MTAP mRNA can be determined both by in situ and by in vitro formats in a biological sample using methods known in the art. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA can be utilized for the purification of RNA from tumor cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155). The isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding MTAP. Other suitable probes for use in the diagnostic assays of the invention are described herein. I-Hybridization of an mRNA with the probe indicates that MTAP gene is being expressed. In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods for use in detecting the level of mRNA encoded by MTAP gene.

An alternative method for determining the level of MTAP mRNA in a sample involves the process of nucleic acid amplification, e.g., by RT-PCR (the experimental embodiment set forth in Mullis, 1987, U.S. Pat. No. 4,683,202), ligase chain reaction (Barany, 1991, Proc. Natl. Acad. Sci. USA, 88:189-193), self-sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the tumor cells prior to detection. In such methods, a cell or tissue sample is prepared/processed using known histological methods. The sample is then immobilized on a support, typically a glass slide, and then contacted with a probe that can hybridize to mRNA that encodes the biomarker.

In another embodiment of the present invention, MTAP protein is detected. A preferred agent for detecting MTAP protein is an antibody capable of binding to MTAP protein or a fragment thereof, preferably an antibody with a detectable label. Antibodies can be polyclonal, or more preferably, monoclonal. An intact antibody, or a fragment or derivative thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

MTAP protein can be isolated from tumor cells using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, N.Y.). A variety of formats can be employed to determine whether a sample contains a protein that binds to a given antibody. Examples of such formats include, but are not limited to, enzyme immunoassay (EIA), radioimmunoassay (RIA), Western blot analysis and enzyme linked immunosorbant assay (ELISA). A skilled artisan can readily adapt known protein/antibody detection methods for use in determining whether tumor cells express a biomarker of the present invention. In one format, antibodies, or antibody fragments or derivatives, can be used in methods such as Western blots or immunofluorescence techniques to detect the expressed MTAP protein. In such uses, it is generally preferable to immobilize either the antibody or MTAP protein on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. One skilled in the art will appreciate that there are many other suitable carriers for binding antibody or antigen, and will be able to adapt such support for use with the present invention. For example, MTAP protein isolated from tumor cells can be run on a polyacrylamide gel electrophoresis and immobilized onto a solid phase support such as nitrocellulose. The support can then be washed with suitable buffers followed by treatment with the delectably labeled antibody. The solid phase support can then be washed with the buffer a second time to remove unbound antibody. The amount of bound label on the solid support can then be detected by conventional means.

For ELISA assays, specific binding pairs can be of the immune or non-immune type. Immune specific binding pairs are exemplified by antigen-antibody systems or hapten/anti-hapten systems. There can be mentioned fluorescein/anti-fluorescein, dinitrophenyl/anti-dinitrophenyl, biotin/anti-biotin, peptide/anti-peptide and the like. The antibody member of the specific binding pair can be produced by customary methods familiar to those skilled in the art. Such methods involve immunizing an animal with the antigen member of the specific binding pair. If the antigen member of the specific binding pair is not immunogenic, e.g., a hapten, it can be covalently coupled to a carrier protein to render it immunogenic. Non-immune binding pairs include systems wherein the two components share a natural affinity for each other but are not antibodies. Exemplary non-immune pairs are biotin-streptavidin, intrinsic factor-vitamin B₁₂, folic acid-folate binding protein and the like.

A variety of methods are available to covalently label antibodies with members of specific binding pairs. Methods are selected based upon the nature of the member of the specific binding pair, the type of linkage desired, and the tolerance of the antibody to various conjugation chemistries. Biotin can be covalently coupled to antibodies by utilizing commercially available active derivatives. Some of these are biotin-N-hydroxy-succinimide which binds to amine groups on proteins; biotin hydrazide which binds to carbohydrate moieties, aldehydes and carboxyl groups via a carbodiimide coupling; and biotin maleimide and iodoacetyl biotin which bind to sulfhydryl groups. Fluorescein can be coupled to protein amine groups using fluorescein isothiocyanate. Dinitrophenyl groups can be coupled to protein amine groups using 2,4-dinitrobenzene sulfate or 2,4-dinitrofluorobenzene. Other standard methods of conjugation can be employed to couple monoclonal antibodies to a member of a specific binding pair including dialdehyde, carbodiimide coupling, homofunctional crosslinking, and heterobifunctional crosslinking. Carbodiimide coupling is an effective method of coupling carboxyl groups on one substance to amine groups on another. Carbodiimide coupling is facilitated by using the commercially available reagent 1-ethyl-3-(dimethyl-aminopropyl)-carbodiimide (EDAC).

Homobifunctional crosslinkers, including the bifunctional imidoesters and bifunctional N-hydroxysuccinimide esters, are commercially available and are employed for coupling amine groups on one substance to amine groups on another. Heterobifunctional crosslinkers are reagents which possess different functional groups. The most common commercially available heterobifunctional crosslinkers have an amine reactive N-hydroxysuccinimide ester as one functional group, and a sulfhydryl reactive group as the second functional group. The most common sulfhydryl reactive groups are maleimides, pyridyl disulfides and active halogens. One of the functional groups can be a photoactive aryl nitrene, which upon irradiation reacts with a variety of groups.

The detectably-labeled antibody or detectably-labeled member of the specific binding pair is prepared by coupling to a reporter, which can be a radioactive isotope, enzyme, fluorogenic, chemiluminescent or electrochemical materials. Two commonly used radioactive isotopes are ¹²⁵I and ³H. Standard radioactive isotopic labeling procedures include the chloramine T, lactoperoxidase and Bolton-Hunter methods for ¹²⁵I and reductive methylation for ³H. The term “detectably-labeled” refers to a molecule labeled in such a way that it can be readily detected by the intrinsic enzymic activity of the label or by the binding to the label of another component, which can itself be readily detected.

Enzymes suitable for use in this invention include, but are not limited to, horseradish peroxidase, alkaline phosphatase, β-galactosidase, glucose oxidase, luciferases, including firefly and renilla, β-lactamase, urease, green fluorescent protein (GFP) and lysozyme. Enzyme labeling is facilitated by using dialdehyde, carbodiimide coupling, homobifunctional crosslinkers and heterobifunctional crosslinkers as described above for coupling an antibody with a member of a specific binding pair.

The labeling method chosen depends on the functional groups available on the enzyme and the material to be labeled, and the tolerance of both to the conjugation conditions. The labeling method used in the present invention can be one of, but not limited to, any conventional methods currently employed including those described by Engvall and Pearlmann, Immunochemistry 8, 871 (1971), Avrameas and Ternynck, Immunochemistry 8, 1175 (1975), Ishikawa et al., J. Immunoassay 4(3):209-327 (1983) and Jablonski, Anal. Biochem. 148:199 (1985). Labeling can be accomplished by indirect methods such as using spacers or other members of specific binding pairs. An example of this is the detection of a biotinylated antibody with unlabeled streptavidin and biotinylated enzyme, with streptavidin and biotinylated enzyme being added either sequentially or simultaneously. Thus, according to the present invention, the antibody used to detect can be detectably-labeled directly with a reporter or indirectly with a first member of a specific binding pair. When the antibody is coupled to a first member of a specific binding pair, then detection is effected by reacting the antibody-first member of a specific binding complex with the second member of the binding pair that is labeled or unlabeled as mentioned above. Moreover, the unlabeled detector antibody can be detected by reacting the unlabeled antibody with a labeled antibody specific for the unlabeled antibody. In this instance “detectably-labeled” as used above is taken to mean containing an epitope by which an antibody specific for the unlabeled antibody can bind. Such an anti-antibody can be labeled directly or indirectly using any of the approaches discussed above. For example, the anti-antibody can be coupled to biotin which is detected by reacting with the streptavidin-horseradish peroxidase system discussed above. In one embodiment of this invention biotin is utilized. The biotinylated antibody is in turn reacted with streptavidin-horseradish peroxidase complex. Orthophenylenediamine, 4-chloro-naphthol, tetramethylbenzidine (TMB), ABTS, BTS or ASA can be used to effect chromogenic detection.

In one immunoassay format for practicing this invention, a forward sandwich assay is used in which the capture reagent has been immobilized, using conventional techniques, on the surface of a support. Suitable supports used in assays include synthetic polymer supports, such as polypropylene, polystyrene, substituted polystyrene, e.g. aminated or carboxylated polystyrene, polyacrylamides, polyamides, polyvinylchloride, glass beads, agarose, or nitrocellulose.

In an aspect of the invention there is provided a kit comprising a reagent for measuring in a tumor sample the expression level of an MTAP gene, the absence of an MTAP gene or reduction of the level or function of MTAP protein, said kit further comprising instructions for administering a therapeutically effective amount of a MAT2A inhibitor. Such kits can be used to determine if a subject is suffering from or is at increased risk of developing a tumor that is less susceptible to inhibition by a MAT2A inhibitors. For example, the kit can comprise a labeled compound or agent capable of detecting MTAP protein or nucleic acid in a biological sample and means for determining the amount of the protein or mRNA in the sample (e.g., an antibody which binds the protein or a fragment thereof, or an oligonucleotide probe which binds to DNA or mRNA encoding the protein). Kits can also include instructions for interpreting the results obtained using the kit. For antibody-based kits, the kit can comprise, for example: (1) a first antibody (e.g., attached to a solid support) which binds to MTAP protein; and, optionally, (2) a second, different antibody which binds to either the protein or the first antibody and is conjugated to a detectable label.

For oligonucleotide-based kits, the kit can comprise, for example: (1) an oligonucleotide, e.g., a detectably labeled oligonucleotide, which hybridizes to a nucleic acid sequence encoding MTAP protein or (2) a pair of primers useful for amplifying MTAP nucleic acid. The kit can also comprise, e.g., a buffering agent, a preservative, or a protein stabilizing agent. The kit can further comprise components necessary for detecting the detectable label (e.g., an enzyme or a substrate). The kit can also contain a control sample or a series of control samples which can be assayed and compared to the test sample. Each component of the kit can be enclosed within an individual container and all of the various containers can be within a single package, along with instructions for interpreting the results of the assays performed using the kit.

The present invention further provides a method for treating tumors in a patient, comprising the steps of diagnosing a patient's likely responsiveness to a MAT2A inhibitor by assessing the MTAP status i.e. whether the expression of the MTAP gene has been reduced, the MTAP gene is absent, or the MTAP protein is absent or of reduced function, by for example any of the methods described herein for determining the expression level of MTAP gene, and administering to said patient a therapeutically effective amount of a MAT2A inhibitor. In this method one or more additional anti-cancer agents or treatments can be co-administered simultaneously or sequentially with the MAT2A inhibitor, as judged to be appropriate by the administering physician given the prediction of the likely responsiveness of the patient to a MTAP inhibitor, in combination with any additional circumstances pertaining to the individual patient.

It will be appreciated by one of skill in the medical arts that the exact manner of administering to said patient of a therapeutically effective amount of a MAT2A inhibitor following a diagnosis of a patient's likely responsiveness to a MAT2A inhibitor will be at the discretion of the attending physician. The mode of administration, including dosage, combination with other anti-cancer agents, timing and frequency of administration, and the like, may be affected by the diagnosis of a patient's likely responsiveness to a MAT2A inhibitor, as well as the patient's condition and history.

In the context of the invention, the MAT2A inhibitor may be administered in combination with cytotoxic, chemotherapeutic or anti-cancer agents, including for example: alkylating agents or agents with an alkylating action, such as cyclophosphamide (CTX; e.g. CYTOXAN®), chlorambucil (CHL; e.g. LEUKERAN®), cisplatin (CisP; e.g. PLATINOL®) busulfan (e.g. MYLERAN®), melphalan, carmustine (BCNU), streptozotocin, triethylenemelamine (TEM), mitomycin C, and the like; anti-metabolites, such as methotrexate (MTX), etoposide (VP16; e.g. VEPESID®), 6-mercaptopurine (6MP), 6-thiocguanine (6TG), cytarabine (Ara-C), 5-fluorouracil (5-FU), capecitabine (e.g. XELODA®), dacarbazine (DTIC), and the like; antibiotics, such as actinomycin D, doxorubicin (DXR; e.g. ADRIAMYCIN®), daunorubicin (daunomycin), bleomycin, mithramycin and the like; alkaloids, such as vinca alkaloids such as vincristine (VCR), vinblastine, and the like; and other antitumor agents, such as paclitaxel (e.g. TAXOL®) and pactitaxel derivatives, the cytostatic agents, glucocorticoids such as dexamethasone (DEX; e.g. DECADRON®) and corticosteroids such as prednisone, nucleoside enzyme inhibitors such as hydroxyurea, amino acid depleting enzymes such as asparaginase, leucovorin and other folic acid derivatives, and similar, diverse antitumor agents. The following agents may also be used as additional agents: arnifostine (e.g. ETHYOL®), dactinomycin, mechlorethamine (nitrogen mustard), streptozocin, cyclophosphamide, lomustine (CCNU), doxorubicin lipo (e.g. DOXIL®), gemcitabine (e.g. GEMZAR®), daunorubicin lipo (e.g. DAUNOXOME®), procarbazine, mitomycin, docetaxel (e.g. TAXOTERE®), aldesleukin, carboplatin, oxaliplatin, cladribine, camptothecin, CPT 11 (irinotecan), 10-hydroxy 7-ethyl-camptothecin (SN38), floxuridine, fludarabine, ifosfamide, idarubicin, mesna, interferon beta, interferon alpha, mitoxantrone, topotecan, leuprolide, megestrol, melphalan, mercaptopurine, plicamycin, mitotane, pegaspargase, pentostatin, pipobroman, plicamycin, tamoxifen, teniposide, testolactone, thioguanine, thiotepa, uracil mustard, vinorelbine, chlorambucil.

The present invention further provides the preceding methods for treating tumors in a patient, comprising administering to the patient a therapeutically effective amount of a MAT2A inhibitor and in addition, simultaneously or sequentially, one or more anti-hormonal agents. As used herein, the term “anti-hormonal agent” includes natural or synthetic organic or peptidic compounds that act to regulate or inhibit hormone action on tumors. Antihormonal agents include, for example: steroid receptor antagonists, anti-estrogens such as tamoxifen, raloxifene, aromatase inhibiting 4(5)-imidazoles, other aromatase inhibitors, 42-hydroxytamoxifen, trioxifene, keoxifene, LY 117018, onapristone, and toremifene (e.g. FARESTON®); anti-androgens such as flutamide, nilutamide, bicalutamide, leuprolide, and goserelin; and pharmaceutically acceptable salts, acids or derivatives of any of the above; agonists and/or antagonists of glycoprotein hormones such as follicle stimulating hormone (FSH), thyroid stimulating hormone (TSH), and luteinizing hormone (LH) and LHRH (leuteinizing hormone-releasing hormone); the LHRH agonist goserelin acetate, commercially available as ZOLADEX® (AstraZeneca); the LHRH antagonist D-alaninamide N-acetyl-3-(2-naphthalenyl)-D-alanyl-4-chloro-D-phenylalanyl-3-(3-pyridinyl)-D-alanyl-L-seryl-N6-(3-pyridinylcarbonyl)-L-lysyl-N6-(3-pyridinylcarbonyl)-D-lysyl-L-leucyl-N6-(1-methylethyl)-L-lysyl-L-proline (e.g ANTIDE®, Ares-Serono); the LHRH antagonist ganirelix acetate; the steroidal anti-androgens cyproterone acetate (CPA) and megestrol acetate, commercially available as MEGACE® (Bristol-Myers Oncology); the nonsteroidal anti-androgen flutamide (2-methyl-N-[4,20-nitro-3-(trifluoromethyl) phenylpropanamide), commercially available as EULEXIN® (Schering Corp.); the non-steroidal anti-androgen nilutamide, (5,5-dimethyl-3-[4-nitro-3-(trifluoromethyl-4′-nitrophenyl)-4,4-dimethyl-imidazolidine-dione); and antagonists for other non-permissive receptors, such as antagonists for RAR, RXR, TR, VDR, and the like.

The use of the cytotoxic and other anticancer agents described above in chemotherapeutic regimens is generally well characterized in the cancer therapy arts, and their use herein falls under the same considerations for monitoring tolerance and effectiveness and for controlling administration routes and dosages, with some adjustments. For example, the actual dosages of the cytotoxic agents may vary depending upon the patient's cultured cell response determined by using histoculture methods. Generally, the dosage will be reduced compared to the amount used in the absence of additional other agents. Typical dosages of an effective cytotoxic agent can be in the ranges recommended by the manufacturer, and where indicated by in vitro responses or responses in animal models, can be reduced by up to about one order of magnitude concentration or amount. Thus, the actual dosage will depend upon the judgment of the physician, the condition of the patient, and the effectiveness of the therapeutic method based on the in vitro responsiveness of the primary cultured malignant cells or histocultured tissue sample, or the responses observed in the appropriate animal models.

The present invention further provides the preceding methods for treating tumors or tumor metastases in a patient, comprising administering to the patient a therapeutically effective amount of a MAT2A inhibitor and in addition, simultaneously or sequentially, one or more angiogenesis inhibitors. Anti-angiogenic agents include, for example: VEGFR inhibitors, such as SU-5416 and SU-6668 (Sugen Inc. of South San Francisco, Calif., USA), or as described in, for example International Application Nos. WO 99/24440, WO 99/62890, WO 95/21613, WO 99/61422, WO 98/50356, WO 99/10349, WO 97/32856, WO 97/22596, WO 98/54093, WO 98/02438, WO 99/16755, and WO 98/02437, and U.S. Pat. Nos. 5,883,113, 5,886,020, 5,792,783, 5,834,504 and 6,235,764; VEGF inhibitors such as IM862 (Cytran Inc. of Kirkland, Wash., USA); angiozyme, a synthetic ribozyme from Ribozyme (Boulder, Colo.) and Chiron (Emeryville, Calif.); and antibodies to VEGF, such as bevacizumab (e.g. AVASTIN™, Genentech, South San Francisco, Calif.), a recombinant humanized antibody to VEGF; integrin receptor antagonists and integrin antagonists, such as to αᵥβ₃. αᵥβ₅ and αᵥβ₆ integrins, and subtypes thereof, e.g. cilengitide (EMD 121974), or the anti-integrin antibodies, such as for example αᵥβ₃ specific humanized antibodies (e.g. VITAXIN®); factors such as IFN-alpha (U.S. Pat. Nos. 41,530,901, 4,503,035, and 5,231,176); angiostatin and plasminogen fragments (e.g. kringle 1-4, kringle 5, kringle 1-3 (O'Reilly, M. S. et al. (1994) Cell 79:315-328; Cao et al. (1996) J. Biol. Chem. 271: 29461-29467; Cao et al. (1997) J. Biol. Chem. 272:22924-22928); endostatin (O'Reilly, M. S. et al. (1997) Cell 88:277; and International Patent Publication No. WO 97/15666); thrombospondin (TSP-1; Frazier, (1991) Curr. Opin. Cell Biol. 3:792); platelet factor 4 (PF4); plasminogen activator/urokinase inhibitors; urokinase receptor antagonists; heparinases; fumagillin analogs such as TNP-4701; suramin and suramin analogs; angiostatic steroids; bFGF antagonists; flk-1 and flt-1 antagonists; anti-angiogenesis agents such as MMP-2 (matrix-metalloproteinase 2) inhibitors and MMP-9 (matrix-metalloproteinase 9) inhibitors. Examples of useful matrix metalloproteinase inhibitors are described in International Patent Publication Nos. WO 96/33172, WO 96/27583, WO 98/07697, WO 98/03516, WO 98/34918, WO 98/34915, WO 98/33768, WO 98/30566, WO 90/05719, WO 99/52910, WO 99/52889, WO 99/29667, and WO 99/07675, European Patent Publication Nos. 818,442, 780,386, 1,004,578, 606,046, and 931,788; Great Britain Patent Publication No. 9912961, and U.S. Pat. Nos. 5,863,949 and 5,861,510. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13).

The present invention further provides the preceding methods for treating tumors in a patient, comprising administering to the patient a therapeutically effective amount of a MAT2A inhibitor and in addition, simultaneously or sequentially, one or more tumor cell pro-apoptotic or apoptosis-stimulating agents. The present invention further provides the preceding methods for treating tumors in a patient, comprising administering to the patient a therapeutically effective amount of a MAT2A inhibitor and in addition, simultaneously or sequentially, one or more signal transduction inhibitors. Signal transduction inhibitors include, for example: erbB2 receptor inhibitors, such as organic molecules, or antibodies that bind to the erbB2 receptor, for example, trastuzumab (e.g. HERCEPTIN®); inhibitors of other protein tyrosine-kinases, e.g. imitinib (e.g. GLEEVEC®); ras inhibitors; raf inhibitors (e.g. BAY 43-9006, Onyx Pharmaceuticals/Bayer Pharmaceuticals); MEK inhibitors; mTOR inhibitors; cyclin dependent kinase inhibitors; protein kinase C inhibitors; and PDK-1 inhibitors (see Dancey, J. and Sausville, E. A. (2003) Nature Rev. Drug Discovery 2:92-313, for a description of several examples of such inhibitors, and their use in clinical trials for the treatment of cancer). ErbB2 receptor inhibitors include, for example: ErbB2 receptor inhibitors, such as GW-282974 (Glaxo Wellcome plc), monoclonal antibodies such as AR-209 (Aronex Pharmaceuticals Inc. of The Woodlands, Tex., USA) and 213-1 (Chiron), and erbB2 inhibitors such as those described in International Publication Nos. WO 98/02434, WO 99/35146, WO 99/35132, WO 98/02437, WO 97/13760, and WO 95/19970, and U.S. Pat. Nos. 5,587,458, 5,877,305, 6,465,449 and 6,541,481.

The present invention further provides the preceding methods for treating tumors in a patient, comprising administering to the patient a therapeutically effective amount of a MAT2A inhibitor and in addition, simultaneously or sequentially, one or more additional anti-proliferative agents. Additional antiproliferative agents include, for example: Inhibitors of the enzyme farnesyl protein transferase and inhibitors of the receptor tyrosine kinase PDGFR, including the compounds disclosed and claimed in U.S. Pat. Nos. 6,080,769, 6,194,438, 6,258,824, 6,586,447, 6,071,935, 6,495,564, 6,150,377, 6,596,735 and 6,479,513, and International Patent Publication WO 01/40217.

The present invention further provides the preceding methods for treating tumors in a patient, comprising administering to the patient a therapeutically effective amount of MAT2A inhibitor and in addition, simultaneously or sequentially, treatment with radiation or a radiopharmaceutical. The source of radiation can be either external or internal to the patient being treated. When the source is external to the patient, the therapy is known as external beam radiation therapy (EBRT). When the source of radiation is internal to the patient, the treatment is called brachytherapy (BT). Radioactive atoms for use in the context of this invention can be selected from the group including, but not limited to, radium, cesium-137, iridium-192, americium-241, gold-198, cobalt-57, copper-67, technetium-99, iodine-123, iodine-131, and indium-111. Where the MAT2A inhibitor according to this invention is an antibody, it is also possible to label the antibody with such radioactive isotopes. Radiation therapy is a standard treatment for controlling unresectable or inoperable tumors and/or tumor metastases. Improved results have been seen when radiation therapy has been combined with chemotherapy. Radiation therapy is based on the principle that high-dose radiation delivered to a target area will result in the death of reproductive cells in both tumor and normal tissues. The radiation dosage regimen is generally defined in terms of radiation absorbed dose (Gy), time and fractionation, and must be carefully defined by the oncologist. The amount of radiation a patient receives will depend on various considerations, but the two most important are the location of the tumor in relation to other critical structures or organs of the body, and the extent to which the tumor has spread. A typical course of treatment for a patient undergoing radiation therapy will be a treatment schedule over a 1 to 6 week period, with a total dose of between 10 and 80 Gy administered to the patient in a single daily fraction of about 1.8 to 2.0 Gy, 5 days a week. In a preferred embodiment of this invention there is synergy when tumors in human patients are treated with the combination treatment of the invention and radiation. In other words, the inhibition of tumor growth by means of the agents comprising the combination of the invention is enhanced when combined with radiation, optionally with additional chemotherapeutic or anticancer agents. Parameters of adjuvant radiation therapies are, for example, contained in International Patent Publication WO 99/60023.

The present invention further provides the preceding methods for treating tumors or tumor metastases in a patient, comprising administering to the patient a therapeutically effective amount of MAT2A inhibitor and in addition, simultaneously or sequentially, treatment with one or more agents capable of enhancing antitumor immune responses. Agents capable of enhancing antitumor immune responses include, for example: CTLA4 (cytotoxic lymphocyte antigen 4) antibodies (e.g. MDX-CTLA4), and other agents capable of blocking CTLA4. Specific CTLA4 antibodies that can be used in the present invention include those described in U.S. Pat. No. 6,682,736.

As used herein, the term “patient” preferably refers to a human in need of treatment with a MAT2A inhibitor for any purpose, and more preferably a human in need of such a treatment to treat cancer, or a precancerous condition or lesion. However, the term “patient” can also refer to non-human animals, preferably mammals such as dogs, cats, horses, cows, pigs, sheep and non-human primates, among others, that are in need of treatment with a MAT2A inhibitor.

The cancer is preferably any cancer treatable, either partially or completely, by administration of MAT2A inhibitor. The cancer may be, for example, lung cancer, non small cell lung (NSCL) cancer, bronchioloalveolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, chronic or acute leukemia, lymphocytic lymphomas, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenomas, including refractory versions of any of the above cancers, or a combination of one or more of the above cancers. The precancerous condition or lesion includes, for example, the group consisting of oral leukoplakia, actinic keratosis (solar keratosis), precancerous polyps of the colon or rectum, gastric epithelial dysplasia, adenomatous dysplasia, hereditary nonpolyposis colon cancer syndrome (HNPCC), Barrett's esophagus, bladder dysplasia, and precancerous cervical conditions.

The MAT2A inhibitor will typically be administered to the patient in a dose regimen that provides for the most effective treatment of the cancer (from both efficacy and safety perspectives) for which the patient is being treated, as known in the art. In conducting the treatment method of the present invention, the MAT2A inhibitor can be administered in any effective manner known in the art, such as by oral, topical, intravenous, intra-peritoneal, intramuscular, intra-articular, subcutaneous, intranasal, intra-ocular, vaginal, rectal, or intradermal routes, depending upon the type of cancer being treated, the type of MAT2A inhibitor being used (for example, small molecule, antibody, RNAi, ribozyme or antisense construct), and the medical judgement of the prescribing physician as based, e.g., on the results of published clinical studies.

The amount of MAT2A kinase inhibitor administered and the timing of administration will depend on the type (species, gender, age, weight, etc.) and condition of the patient being treated, the severity of the disease or condition being treated, and on the route of administration. For example, small molecule MAT2A inhibitors can be administered to a patient in doses ranging from 0.001 to 100 mg/kg of body weight per day or per week in single or divided doses, or by continuous infusion. Antibody-based MAT2A inhibitors, or antisense, RNAi or ribozyme constructs, can be administered to a patient in doses ranging from 0.1 to 100 mg/kg of body weight per day or per week in single or divided doses, or by continuous infusion. In some instances, dosage levels below the lower limit of the aforesaid range may be more than adequate, while in other cases still larger doses may be employed without causing any harmful side effect, provided that such larger doses are first divided into several small doses for administration throughout the day.

The MAT2A inhibitor can be administered with various pharmaceutically acceptable inert carriers in the form of tablets, capsules, lozenges, troches, hard candies, powders, sprays, creams, salves, suppositories, jellies, gels, pastes, lotions, ointments, elixirs, syrups, and the like. Administration of such dosage forms can be carried out in single or multiple doses. Carriers include solid diluents or fillers, sterile aqueous media and various non-toxic organic solvents, etc. Oral pharmaceutical compositions can be suitably sweetened and/or flavored. The inhibitor can be combined together with various pharmaceutically acceptable inert carriers in the form of sprays, creams, salves, suppositories, jellies, gels, pastes, lotions, ointments, and the like. Administration of such dosage forms can be carried out in single or multiple doses. Carriers include solid diluents or fillers, sterile aqueous media, and various non-toxic organic solvents, etc. All formulations comprising proteinaceous inhibitors should be selected so as to avoid denaturation and/or degradation and loss of biological activity of the inhibitor.

Methods of preparing pharmaceutical compositions comprising a MAT2A inhibitor are known in the art, and for example are described, in Remington's Pharmaceutical Sciences, Mack Publishing Company, Easton, Pa., 18^{th} edition (1990). For oral administration of inhibitors, tablets containing one or both of the active agents are combined with any of various excipients such as, for example, micro-crystalline cellulose, sodium citrate, calcium carbonate, dicalcium phosphate and glycine, along with various disintegrants such as starch (and preferably corn, potato or tapioca starch), alginic acid and certain complex silicates, together with granulation binders like polyvinyl pyrrolidone, sucrose, gelatin and acacia. Additionally, lubricating agents such as magnesium stearate, sodium lauryl sulfate and talc are often very useful for tableting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the inhibitor may be combined with various sweetening or flavoring agents, coloring matter or dyes, and, if so desired, emulsifying and/or suspending agents as well, together with such diluents as water, ethanol, propylene glycol, glycerin and various like combinations thereof. For parenteral administration of either or both of the active agents, solutions in either sesame or peanut oil or in aqueous propylene glycol may be employed, as well as sterile aqueous solutions comprising the active agent or a corresponding water-soluble salt thereof. Such sterile aqueous solutions are preferably suitably buffered, and are also preferably rendered isotonic, e.g., with sufficient saline or glucose. These particular aqueous solutions are especially suitable for intravenous, intramuscular, subcutaneous and intraperitoneal injection purposes. The oily solutions are suitable for intra-articular, intramuscular and subcutaneous injection purposes. The preparation of all these solutions under sterile conditions is readily accomplished by standard pharmaceutical techniques well known to those skilled in the art. Any parenteral formulation selected for administration of proteinaceous inhibitors should be selected so as to avoid denaturation and loss of biological activity of the inhibitor.

Additionally, it is possible to topically administer either or both of the active agents, by way of, for example, creams, lotions, jellies, gels, pastes, ointments, salves and the like, in accordance with standard pharmaceutical practice. For example, a topical formulation comprising a MAT2A inhibitor in about 0.1% (w/v) to about 5% (w/v) concentration can be prepared.

For veterinary purposes, the active agents can be administered separately or together to animals using any of the forms and by any of the routes described above. In a preferred embodiment, the inhibitor is administered in the form of a capsule, bolus, tablet, liquid drench, by injection or as an implant. As an alternative, the inhibitor can be administered with the animal feedstuff, and for this purpose a concentrated feed additive or premix may be prepared for a normal animal feed. Such formulations are prepared in a conventional manner in accordance with standard veterinary practice.

Techniques for the production and isolation of monoclonal antibodies and antibody fragments are well-known in the art, and are described in Harlow and Lane, 1988, Antibodies: A Laboratory Manual, Cold Spring Harbor Laboratory, and in J. W. Goding, 1986, Monoclonal Antibodies: Principles and Practice, Academic Press, London. Humanized anti-MAT2A antibodies and antibody fragments can also be prepared according to known techniques such as those described in Vaughn, T. J. et al., 1998, Nature Biotech. 16:535-539 and references cited therein, and such antibodies or fragments thereof are also useful in practicing the present invention.

MAT2A inhibitors for use in the present invention can alternatively be based on antisense oligonucleotide constructs. Anti-sense oligonucleotides, including anti-sense RNA molecules and anti-sense DNA molecules, would act to directly block the translation of MAT2A mRNA by binding thereto and thus preventing protein translation or increasing mRNA degradation, thus decreasing the level MAT2A protein, and thus activity, in a cell. For example, antisense oligonucleotides of at least about 15 bases and complementary to unique regions of the mRNA transcript sequence encoding MAT2A can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors for use in the present invention. MAT2A gene expression can be reduced by contacting the tumor, subject or cell with a small double stranded RNA (dsRNA), or a vector or construct causing the production of a small double stranded RNA, such that expression of MAT2A is specifically inhibited (i.e. RNA interference or RNAi). Methods for selecting an appropriate dsRNA or dsRNA-encoding vector are well known in the art for genes whose sequence is known (e.g. see Tuschi, T., et al. (1999) Genes Dev. 13(24):3191-3197; Elbashir, S. M. et al. (2001) Nature 411:494-498; Hannon, G. J. (2002) Nature 418:244-251; McManus, M. T. and Sharp, P. A. (2002) Nature Reviews Genetics 3:737-747; Bremmelkamp, T. R. et al. (2002) Science 296:550-553; U.S. Pat. Nos. 6,573,099 and 6,506,559; and International Patent Publication Nos. WO 01/36646, WO 99/32619, and WO 01/68836).

Ribozymes can also function as inhibitors for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as inhibitors can be prepared by known methods. These include techniques for chemical synthesis such as, e.g., by solid phase phosphoramidite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

The term “pharmaceutically acceptable salts” refers to salts prepared from pharmaceutically acceptable non-toxic bases or acids. When a compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (cupric and cuprous), ferric, ferrous, lithium, magnesium, manganese (manganic and manganous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropyl amine, lysine, methylglucamine, morpholine, piperazine, piperidine, polyamine resins, procaine, purines, theobromine, triethylameine, trimethylamine, tripropylamine, tromethamine and the like.

When a compound used in the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric and tartaric acids.

Pharmaceutical compositions used in the present invention comprising a MAT2A inhibitor compound (including pharmaceutically acceptable salts thereof) as active ingredient, can include a pharmaceutically acceptable carrier and optionally other therapeutic ingredients or adjuvants. Other therapeutic agents may include those cytotoxic, chemotherapeutic or anti-cancer agents, or agents which enhance the effects of such agents, as listed above. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the inhibitor compounds (including pharmaceutically acceptable salts thereof) of this invention can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, a MAT2A inhibitor compound (including pharmaceutically acceptable salts of each component thereof) may also be administered by controlled release means and/or delivery devices. The combination compositions may be prepared by any of the methods of pharmacy. In general, such methods include a step of bringing into association the active ingredients with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

An inhibitor compound (including pharmaceutically acceptable salts thereof) used in this invention, can also be included in pharmaceutical compositions in combination with one or more other therapeutically active compounds. Other therapeutically active compounds may include those cytotoxic, chemotherapeutic or anti-cancer agents, or agents which enhance the effects of such agents, as listed above. Thus in one embodiment of this invention, the pharmaceutical composition can comprise a MAT2A inhibitor compound in combination with an anticancer agent, wherein said anti-cancer agent is a member selected from the group consisting of alkylating drugs, antimetabolites, microtubule inhibitors, podophyllotoxins, antibiotics, nitrosoureas, hormone therapies, kinase inhibitors, activators of tumor cell apoptosis, and antiangiogenic agents. The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen. In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques. A tablet containing the composition used for this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably contains from about 0.05 mg to about 5 g of the active ingredient. For example, a formulation intended for the oral administration to humans may contain from about 0.5 mg to about 5 g of active agent, compounded with an appropriate and convenient amount of carrier material that may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions used in the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms. Pharmaceutical compositions used in the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof. Pharmaceutical compositions for the present invention can be in a form suitable for topical sue such as, for example, an aerosol, cream, ointment, lotion, dusting powder, or the like. Further, the compositions can be in a form suitable for use in transdermal devices. These formulations may be prepared, utilizing a MAT2A inhibitor compound (including pharmaceutically acceptable salts thereof), via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions for this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds. In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient, Compositions containing a MAT2A inhibitor compound (including pharmaceutically acceptable salts thereof) may also be prepared in powder or liquid concentrate form.

Dosage levels for the compounds used for practicing this invention will be approximately as described herein, or as described in the art for these compounds. It is understood, however, that the specific dose level for any particular patient will depend upon a variety of factors including the age, body weight, general health, sex, diet, time of administration, route of administration, rate of excretion, drug combination and the severity of the particular disease undergoing therapy.

Many alternative experimental methods known in the art may be successfully substituted for those specifically described herein in the practice of this invention, as for example described in many of the excellent manuals and textbooks available in the areas of technology relevant to this invention (e.g. Using Antibodies, A Laboratory Manual, edited by Harlow, E. and Lane, D., 1999, Cold Spring Harbor Laboratory Press, (e.g. ISBN 0-87969-544-7); Roe B. A. et. al. 1996, DNA Isolation and Sequencing (Essential Techniques Series), John Wiley & Sons. (e.g ISBN 0-471-97324-0); Methods in Enzymology: Chimeric Genes and Proteins”, 2000, ed. J. Abelson, M. Simon, S. Emr, J. Thorner. Academic Press; Molecular Cloning: a Laboratory Manual, 2001, 3^{rd} Edition, by Joseph Sambrook and Peter MacCallum, (the former Maniatis Cloning manual) (e.g. ISBN 0-87969-577-3); Current Protocols in Molecular Biology, Ed. Fred M. Ausubel, et. al. John Wiley & Sons (e.g. ISBN 0-471-50338-X); Current Protocols in Protein Science, Ed. John E. Coligan, John Wiley & Sons (e.g. ISBN 0-471-11184-8); and Methods in Enzymology: Guide to protein Purification, 1990, Vol. 182, Ed. Deutscher, M. P., Acedemic Press, Inc. (e.g. ISBN 0-12-213585-7)), or as described in the many university and commercial websites devoted to describing experimental methods in molecular biology.

### REFERENCES

Angermayr, M., Roidl, A., and Bandlow, W. (2002). Yeast Rio1p is the founding member of a novel subfamily of protein serine kinases involved in the control of cell cycle progression. Molecular microbiology 44, 309-324.
Antonysamy, S., Bonday, Z., Campbell, R. M., Doyle, B., Druzina, Z., Gheyi, T., Han, B., Jungheim, L. N., Qian, Y., Rauch, C., et al. (2012). Crystal structure of the human PRMT5:MEP50 complex. Proceedings of the National Academy of Sciences of the United States of America 109, 17960-17965.
Basu, I., Locker, J., Cassera, M. B., Belbin, T. T J., Merino, E. F., Dong, X., Hemeon, I., Evans, G. B., Guha, C., and Schramm, V. L. (2011). Growth and metastases of human lung cancer are inhibited in mouse xenografts by a transition state analogue of 5′-methylthioadenosine phosphorylase. The Journal of biological chemistry 286, 4902-4911.
Chan-Penebre, E., Kuplast, K. G., Majer, C. R., Boriack-Sjodin, P. A., Wigle, T. J., Johnston, L. D., Rioux, N., Munchhof; M. J., Jin, L., Jacques, S. L., et al. (2015) A selective inhibitor of PRMT5 with in vivo and in vitro potency in MCL models. Nature chemical biology 11, 432-437.
Guderian, G., Peter, C., Wiesner, J., Sickmann, A., Schulze-Osthoff, K., Fischer, U., and Grimmler, M. (2011). RioK1, a new interactor of protein arginine methyltransferase 5 (PRMT5), competes with pICln for binding and modulates PRMT5 complex composition and substrate specificity. The Journal of biological chemistry 286, 1976-1986.
Lacroix, M., El Messaoudi, S., Rodier, G., Le Cam, A., Sardet, C., and Fabbrizio, E. (2008). The histone-binding protein COPR5 is required for nuclear functions of the protein arginine methyltransferase PRMT5. EMBO Rep 9, 452-458.
Longshaw, A. I., Adanitsch, F., Gutierrez, J. A., Evans, G. B., Tyler, P. C., and Schramm, V. L. (2010). Design and synthesis of potent “sulfur-free” transition state analogue inhibitors of 5′-methylthioadenosine nucleosidase and 5′-methylthioadenosine phosphorylase. J Med Chem 53, 6730-6746.
Meister, G., Eggert, C., Buhler, D., Brahms, H., Kambach, C., and Fischer, U. (2001). Methylation of Sm proteins by a complex containing PRMT5 and the putative U snRNP assembly factor pICln. Current biology: CB 11, 1990-1994.
Pal, S., Vishwanath, S. N., Erdjument-Bromage, H., Tempst, P., and Sif, S. (2004). Human SWI/SNF-associated PRMT5 methylates histone H3 arginine 8 and negatively regulates expression of ST7 and NM23 tumor suppressor genes. Molecular and cellular biology 24, 9630-9645.
Pesiridis, G. S., Diamond, E., and Van Duyne, G D. (2009). Role of pICLn in methylation of Sm proteins by PRMT5. The Journal of biological chemistry 284, 21347-21359.
Pollack, B. P., Kotenko, S. V., He, W., Izotova, L. S., Barnoski, B. L., and Pestka, S. (1999). The human homologue of the yeast proteins Skb1 and Hsl7p interacts with Jak kinases and contains protein methyltransferase activity. The Journal of biological chemistry 274, 31531-31542.
Sun, L, Wang, M., Lv, Z., Yang, N., Liu, Y., Bao, S., Gong, W., and Xu, R. M. (2011). Structural insights into protein arginine symmetric dimethylation by PRMT5. Proceedings of the National Academy of Sciences of the United States of America 108, 20538-20543.
Widmann, B., Wandrey, F., Badertscher, L., Wyler, E., Pfannstiel, J., Zemp, I., and Kutay, U. (2012). The kinase activity of human Rio1 is required for final steps of cytoplasmic maturation of 40S subunits. Molecular biology of the cell 23, 22-35.
Wiederschain, D., Wee, S., Chen, L., Loo, A., Yang, G., Huang, A., Chen, Y., Caponigro, G., Yao, Y. M., Lengauer, C., et al. (2009). Single-vector inducible lentiviral RNAi system for oncology target validation. Cell cycle 8, 498-504
Wilczek, C., Chitta, R., Woo, E., Shabanowitz, J., Chait, B. T., Hunt, D. F., and Shechter, D. (2011). Protein arginine methyltransferase Prmt5-Mep50 methylates histones H2A and H4 and the histone chaperone nucleoplasrnin in Xenopus laevis eggs. The Journal of biological chemistry 286, 42221-42231.

### EXAMPLES

This invention will be better understood from the Examples that follow. However, one skilled in the art will readily appreciate that the specific methods and results discussed are merely illustrative of the invention as described more fully in the claims which follow thereafter, and are not to be considered in any way limited thereto.

### Cell Lines

HCT116 colon carcinoma MTAP wt and MTAP^{-/-} isogenic cell lines were licensed from Horizon Discovery. All other cell lines were obtained from American Type Culture Collection (ATCC), RIKEN Bioresource Center cell bank, or DSMZ.

shRNA-Based Genomic Screen

An shRNA library comprising 50,468 shRNA targeting 6317 genes was prepared by Cellecta, Inc, by on-chip DNA synthesis, and subsequently cloned into the pRS116-U6-sh-13kCB22-HTS6-UbiC-TagRFP-2A-Puro vector (hGW Module 1 library available from Cellecta, Inc). Lentiviral vector preparation, titering and transduction of HCT116-MTAP−/− and HCT116 MTAP WT cells was conducted as per vendor shRNA Library Screening Reference Manual, v2a (www.cellecta.com) and (Kampmann and Weissman Nature Protocols 2014). shRNA library barcode inserts were amplified by 2-round PCR and sequenced using Illumina Hiseq 2000. All reads with exact match to a library barcode were included in data analysis.

Generation of Stable Inducible shRNA and cDNA Rescue Cell Lines.

All shRNAs constructs were cloned into the pLKO-Tet-on lentiviral backbone vector (Wiederschain et al., 2009). MTAP, PRMT5, Mat2a, and RIOK1 wt and catalytically dead mutant cDNAs were cloned into in pLVX-IRES-neo/puro/blast lentiviral vector. Specific sequences targeted were:

shNT:
5′-CAACAAGATGAAGAGCACCAA-3′
shPRMT5:
5′-GGATAAAGCTGTATGCTGT-3′
shMat2a:
5′-CAGTTTAATGAAGATCTAAAT-3′
shMat2a1:
5′-CTTGTGAAACTGTTGCTAA-3′
shRIOK1:
5′-GTCATGAGTTTCATTGGTAAA-3′

All constructs were confirmed by sequencing. Lentivirus-based (shRNAs or cDNA overexpression) constructs were made using the standard TRC protocol from the Broad Institute (http://www.broadinstitute.org/rnai/public/resources/protocols). Following viral transduction, shRNA or cDNA-expressing cell pools were selected with appropriate drug (puromycin, neomycin blasticidin).
siRNA Transfections

Cells were transfected with ON-Target plus SMARTpool siRNAs (Dharmacon) using Lipofectamine RNAiMAX (13778-150, Life Technologies) per vendor protocol. To ensure robust and durable knock-down of target, two sequential transfections were performed, separated by 24 hours of recovery in full growth media (RPMI+10% FBS). 24 hours after the second transfection, cells were trypsinized, counted, and plated for 96 well format growth assays.

### Growth Assays

Following siRNA transfection or 4-day pre-treatment with 200 ng/ml doxycycline as relevant, cells were plated in 96-well tissue culture plates at 2000 or 3000 cells per well. Cell titer glo ATP assay (Promega) was performed on parallel assay plates at t₀ and at the end of cell culture period as indicated in Figure Legends. Percent growth was calculated as percent change in t_{end}/t₀. For colony formation assays, cells were plated at 1,000 per well of a 6-well plate and doxycycline treatment (200 ng/ml) was initiated at the time of plating using equivalent volume of sterile water as vehicle control. Colonies were fixed after 10 days and stained with 0.05% crystal violet in 4.5% paraformaldehyde solution for 24h. Colonies were quantified using Li-Cor image processing software (Li-Cor Bisciences, Lincoln Nebr.).

### Immunoblotting

Antibodies used were PRMT5 (2252S, Cell Signaling Technology), Mat2a (sc-166452, Santa Cruz Biotechnology), MTAP (sc-100782, Santa Cruz Biotechnology), H4R3me2s (A-3718, Epigentek), histone H4 (ab10158, abcam), eIF4E (9742, Cell Signaling) RIOK1 (A302-456A, Bethyl Laboratories, Inc.), β-actin (3700S, Cell Signaling Technology). Secondary antibodies used were IRDye 680RD Donkey anti-Rabbit (926-68073, LI-COR) and IRDye 800CW Donkey anti-Mouse (926-32212, LI-COR).

### N-Methyltransferase In Vitro Activity Assays

In vitro screening of methyltransferase inhibition by MTA, as well as SAM Km measurements, was conducted using a panel of methyltransferase assays at Eurofins CEREP Panlabs.

### Metabolite Extraction and Targeted LC-MS Analysis

For media analysis, conditioned media was collected from cells that were cultured for at least 24 hr and diluted 20-fold prior to LC-MS analysis. For intracellular metabolites, organic extraction was performed with cold 80/20 (v/v) methanol/water with d⁸-putrescine added as an internal standard following normalization to cell number (100,000 cells per sample were analyzed). Samples were then dried under reduced pressure and stored at −80° C. until LC-MS analysis.

Snap frozen tumors were extracted with 80/20 MeOH/water (v/v) containing d⁸-putrescine internal standard following normalization to weight (mg). Tumor samples were homogenized using the tissue lyser at the maximum frequency for 1 min. Homogenized samples were centrifuged at 14,000 RPMs for 15 minutes at 4° C. A volume of supernatant equivalent to 2 mg of tissue per well was evaporated under reduced pressure, and stored at −80° C. until LC-MS analysis. Prior to injection, dried extracts were reconstituted in LC-MS grade water with 0.1% formic acid.

The extracted samples were analyzed using quantitative liquid chromatography/mass spectrometry on a QExactive orbitrap mass spectrometer (Thermo Fisher Scientific, San Jose, Calif.) as previously described (Jha et al., 2015). Briefly, a Thermo Accela 1250 pump delivered a gradient of 0.025% heptafluorobutyric acid, 0.1% formic acid in water and acetonitrile at 400 μL/min. Stationary phase was an Atlantis T3, 3 μm, 2.1×150 mm column. A QExactive Mass Spectrometer was used at 70,000 resolving power to acquire data in full-scan mode. Data analysis was conducted in MAVEN (Melamud et al., 2010) and Spotfire. Quantitation was performed using an external calibration curve.

### MAT2A Inhibition in Colon Cancer Xenograft

To investigate the effect of MAT2A inhibition in vivo, xenografts with HCT116 isogenic cell lines expressing inducible MAT2A shRNA were prepared. Tumors were allowed to form prior to treatment of animals with doxycycline, to assess the role of MAT2A in proliferation of established tumors. Efficiency of MAT2A knockdown in vivo was confirmed by western blot. MAT2A genetic ablation in vivo was confirmed to reduce SAM levels in HCT116 xenografts of both MTAP^{-/-} and wt MTAP genotypes. To demonstrate selective growth inhibition in vivo was an on-target effect, an expanded in vivo study was performed with a wild type MAT2A rescue arm of shMAT2A. This experiment confirmed the efficacy observed in our first in vivo study and, as with the in vitro studies, growth inhibition was rescued in the xenograft expressing a MAT2A cDNA that was resistant to the MAT2A shRNA.

Tumor Cell Growth Inhibition with MAT2A Inhibitors AG-512 and AG-673

AG-512 and AG-673 are small molecule inhibitors of MAT2A enzymatic activity with IC₅₀ of 83 nM and 143 nM respectively in a biochemical assay and inhibited the production of SAM in cells with IC50s of 80 and 490 nM respectively.

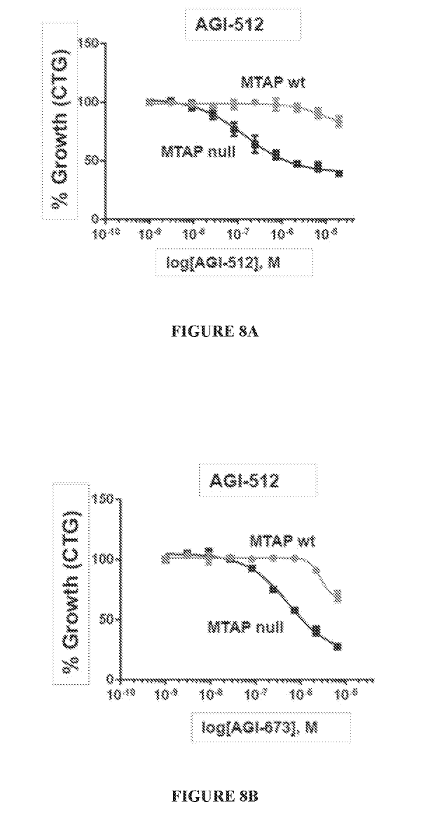

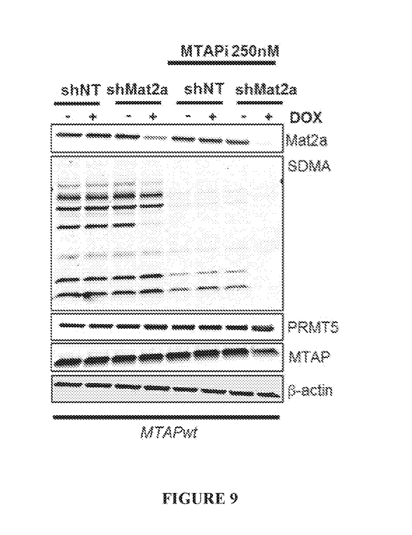

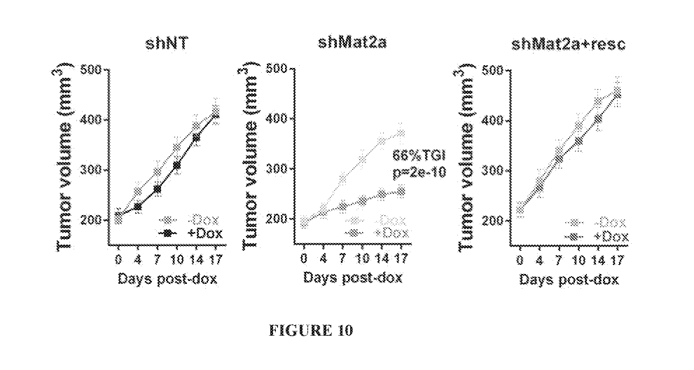

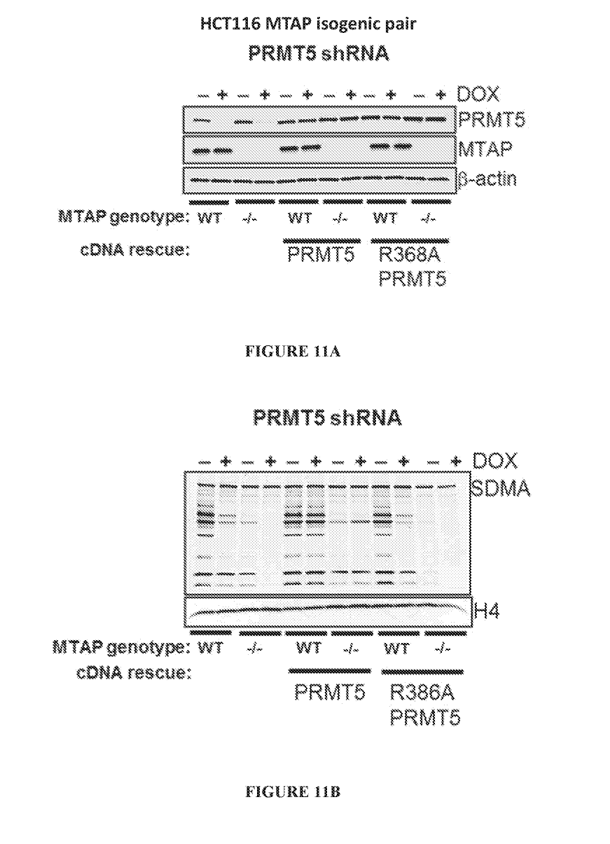

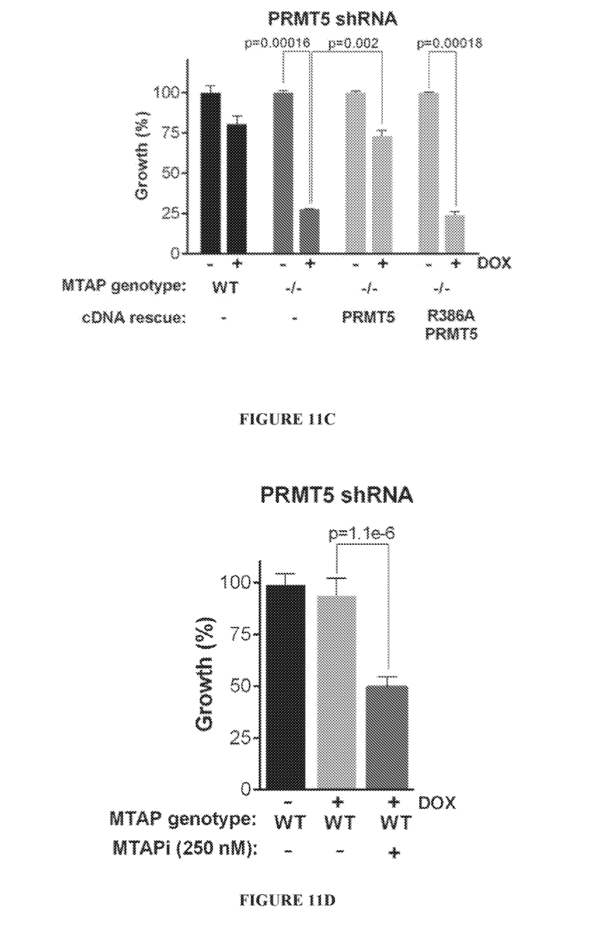

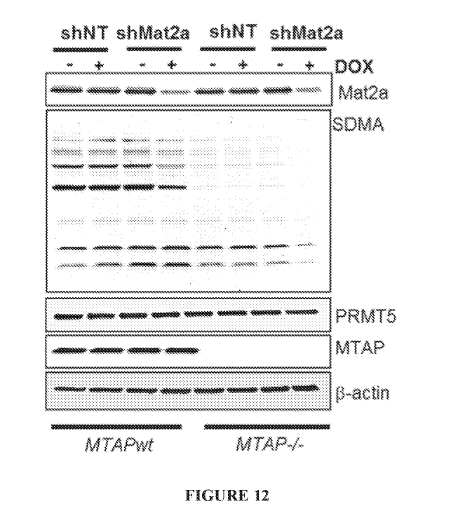

An isogenic clone of HCT116 cells was genetically modified to delete exon 6 of the MTAP gene leading to complete loss of MTAP expression was compared to parental HCT116 cells. Cells were grown in 96-well plates and treated for 4 days with MAT2A inhibitors AG-512 and AG-673. % growth was determined by measuring ATP levels in wells at day 4 vs a control plate that was assayed at day 0 (ie time of initial drug treatment). As shown in FIG. 8A, AG-512 inhibited tumor cell growth of wt MTAP cells with an IC₅₀ of 8.98 μM but with an IC₅₀ of 143 nM in MTAP null cells. Similarly, AG-673 inhibited wt MTAP cells with an IC₅₀ of 2.76 μM and MTAP null cells with an IC₅₀ of 552 nM. More than 50 small molecule inhibitors having diverse chemical structure were observed to inhibit growth of MTAP-null tumor cells which correlated with potency of the compounds to reduce SAM levels.

### MAT2A Inhibition in Cell Line Panel

332 cell lines (68 MTAP null, 224 MTAP wt) were grown in 96-well plates and treated for 6 days with a dose range of MAT2A inhibitor or AGI-673 Percent (%) growth for each dose point was calculated, and curve fit used to determine GI₅₀ (concentration of drug that leads to 50% reduction in growth). Using a GI₅₀<2 μM as a threshold for sensitivity, 36 of 68 (53%) of MTAP null cell lines were sensitive to AGI-673 inhibition while only 34 of 224 (15%) of MTAP wt cell lines were sensitive. (p:=2e-9). Further genomic analysis revealed that in 16 MTAP null cell lines that also incorporate a KRAS mutation (G12X or G13X) 14 (88%) were sensitive to MAT2A inhibition with AGI-673 while only 24 of 49 (49%) of MTAP wild type cell lines were sensitive when a KRAS mutation was present (p.008).

### INCORPORATION BY REFERENCE

All patents, published patent applications and other references disclosed herein are hereby expressly incorporated herein by reference.

### EQUIVALENTS

Those skilled in the art will recognize, or be able to ascertain, using no more than routine experimentation, many equivalents to specific embodiments of the invention described specifically herein. Such equivalents are intended to be encompassed in the scope of the following claims.

1. A method of treating an MTAP null cancer in a subject comprising administering to the subject a therapeutically effective amount of a MAT2A inhibitor.

2. The method of claim 1, further comprising detecting the absence of the MTAP gene in the cancer, e.g. from a sample of the cancer taken from the patient.

3. The method of claim 1, wherein said cancer incorporates a KRAS mutation.

4. The method of claim 1 or, wherein said cancer incorporates a p53 mutation.

5. A method for determining whether survival or proliferation of a tumor cell can be inhibited by contacting said tumor cell with a MAT2A inhibitor, said method comprising determining the status of MTAP in said tumor cell, wherein the reduction or absence MTAP expression or absence of the MTAP gene or reduced level or function of MTAP protein indicates survival or proliferation of said tumor cell can be inhibited by a MAT2A inhibitor.

6. The method of claim 5, wherein the absence of the MTAP gene is determined.

7. The method of claim 5 further comprising determining the presence of a KRAS mutation, wherein the reduction or absence MTAP expression or absence of the MTAP gene or reduced level or function of MTAP protein and the presence of a KRAS mutation indicates survival or proliferation of said tumor cell can be inhibited by a MAT2A inhibitor.

8. The method of claim 5 further comprising determining the presence of a p53 mutation, wherein the reduction or absence MTAP expression or absence of the MTAP gene or reduced level or function of MTAP protein and the presence of a p53 mutation indicates survival or proliferation of said tumor cell can be inhibited by a MAT2A inhibitor

9. A method for characterizing a tumor cell comprising measuring in said tumor cell the level of MTAP gene expression, detecting the presence or absence of an MTAP gene or measuring the level of MTAP protein present, wherein the reduction or absence MTAP expression or absence of the MTAP gene or reduced level or function of MTAP protein relative to a reference cell indicates that survival or proliferation of said tumor cell can be inhibited by a MAT2A inhibitor.

10. The method of claim 9, wherein the absence of MTAP gene in said tumor cell is detected.

11. The method of claim 9, further comprising detecting the presence of a KRAS mutation, wherein the reduction or absence MTAP expression or absence of the MTAP gene or reduced level or function of MTAP protein and the presence of a KRAS mutation indicates that survival or proliferation of said tumor cell can be inhibited by a MAT2A inhibitor.

12. The method of claim 9, further comprising detecting the presence of a p53 mutation, wherein the reduction or absence MTAP expression or absence of the MTAP gene or reduced level or function of MTAP protein and the presence of a p53 mutation indicates that survival or proliferation of said tumor cell can be inhibited by a MAT2A inhibitor.

13. A kit comprising reagents for measuring in a tumor sample the expression level of an MTAP gene, the absence of an MTAP gene or reduction of the level or function of MTAP protein, said kit further comprising instructions for administering a therapeutically effective amount of a MAT2A inhibitor.

14. The kit of claim 13, wherein the reagent are for detecting the absence of MTAP gene in the sample.

15. The kit of claim 13, wherein the kit further includes reagents for detecting the presence of a KRAS mutation.

16. The kit of claim 13, wherein the kit further includes reagents for detecting the presence of a p53 mutation.

17. The method of any one of claims 3, 7, and 11, wherein said KRAS mutation is a G12X or G13X amino acid substitution.

18. The method of claim 17, wherein said KRAS mutation is G12C, G12D G12R, G12V, or G13D.

19. The method of claim 4, 8, and 12, wherein said p53 mutation is, Y126_splice, K132Q, M133K, R174fs, R175H, R196*, C238S, C242Y, G245S, R248W, R248Q, 1255T, D259V, S261_splice, R267P, R273C, R282W, A159V or R280K.

20. The method of any one of claims 1, 5, and 9, wherein said MAT2A inhibitor is a compound of the formula:
X—Ar₁—CR^{a}═CR^{b}—Ar₂
where
R^{a} and R^{b} are independently H, alkyl, halo, alkoxy, cyano;
X represents at least one halogen on Ar₁;
each of Ar₁ and Ar₂ is selected from aryl and heteroaryl, which can be further substituted with halo, amino, alkylamino, dialkylamino, arylalkylamino, N-oxides of dialkylamino, trialkylammonium, mercapto, alkylthio, alkanoyl, nitro, nitrosyl, cyano, alkoxy, alkenyloxy, aryl, heteroaryl, sulfonyl, sulfonamide, CONR₁₁R₁₂, NR₁₁CO(R₁₃), NR₁₁COO(R₁₃), and R₁₁CONR₁₂R₁₃, where R₁₁, R₁₂, R₁₃ are independently selected from H, alkyl, aryl, heteroaryl and a fluorine;
provided that Ar₂ contains at least one nitrogen atom in the aryl ring or at least one nitrogen substituent on the aryl ring.

21. The method of claim 20, wherein said MAT2A inhibitor is a compound of formula: 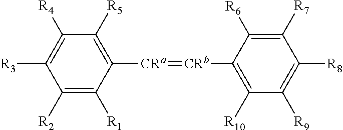
where
R^{a} and R^{b} are as defined above,
R₁ to R₁₀ are independently H, halo, amino, alkylamino, dialkylamino, N-oxides of dialkylamino, arylalkylamino, dialkyloxyamino, trialkylammonium, mercapto, alkylthio, alkanoyl, nitro, nitrosyl, cyano, alkoxy, alkenyloxy, aryl, heteroaryl, sulfonyl, sulfonamide, CONR₁₁R₁₂, NR₁₁CO(R₁₃), NR₁₁COO(R₁₃), and NR₁₁CONR₁₂R₁₃
where R₁₁, R₁₂, R₁₃, are independently selected from H, alkyl, aryl, heteroaryl and a fluorine;
provided at least one of R₁ to R₅ is a halogen, and at least one of R₆ to R₁₀ is a nitrogen containing substituent,
or a pharmaceutically acceptable salt thereof, or a biotinylated derivative thereof.

22. The method of claim 21, wherein the MAT2A inhibitor is selected from the group consisting of: (E)-4-(2-Fluorostyryl)-N,N-dimethylaniline; (E)-4-(3-Fluorostyryl)-N,N-dimethylaniline; (E)-4-(4-Fluorostyryl)-N,N-dimethylaniline; (E)-4-(2-Fluorostyryl)-N,N-diethylaniline; (E)-4-(2-Fluorostyryl)-N,N-di phenylaniline; (E)-1-(4-(2-Fluorostyryl)phenyl)-4-methylpiperazine; (E)-4-(2-Fluorostyryl)-N,N-dimethylnaphthalen-1-amine; (E)-2-(4-(2-Fluorostyryl)phenyl)-1-methyl-1H-imidazole; (E)-4-(2,3-Difluorostyryl)-N,N-dimethylaniline; (E)-4-(2,4-Difluorostyryl)-N,N-dimethylaniline; (E)-4-(2,5-Difluorostyryl)-N,N-dimethylaniline; (E)-2-(2,6-Difluorostyryl)-N,N-dimethylaniline; (E)-3-(2,6-Difluorostyryl)-N,N-dimethyl aniline; (E)-4-(2,6-Difluorostyryl)-N,N-dimethylaniline; (E)-4-(2,6-Difluorostyryl)-N,N-diethylaniline; (E)-4-(3,4-Difluorostyryl)-N,N-dimethylaniline; (E)-4-(3,5-Difluorostyryl)N, N-dimethylaniline; (E)-N,N-Dimethyl-4-(2,3,6-trifluorostyryl)aniline; (E)-N,N-Dimethyl-4-(2,4,6-trifluorostyryl)aniline; (E)-4-(2-chloro-6-fluorostyryl)-N,N-dimethylaniline; (E)-4-(2,6-dichlorostyryl)-N,N-dimethylaniline; (E)-4-(2,6-Difluorophenethyl)-N,N-dimethylaniline; and (E)-2-benzamide-4-(2,6-difluorostyryl)-N,N-dimethylaniline.
